(19) United States
(12) Patent Application Publication
(10) Pub. No.: US 2009/0088545 A1
(43) Pub. Date: Apr. 2, 2009

(54) Polymerziable liquid crystal compound, polymerziable liquid crystal composition comprising the same, and polymer produced using them

(76) Inventors: Daniel Antonio Sahade, Chiba (JP); Takuro Oda, Chiba (JP)

Correspondence Address: BIRCH STEWART KOLASCH & BIRCH, PO BOX 747, FALLS CHURCH, VA, 22040-0747, US

(21) Appl. No.: 11/918,788

(22) PCT Filed: Apr. 18, 2006
(86) PCT No.: PCT/JP2006/308100

§ 371 Date: Oct. 18, 2007

(30) Foreign Application Priority Data
Apr. 20, 2005 (JP) 2005-121800

(51) Int. Cl. C08F 24/00; C07D 307/32

(52) U.S. Cl. 526/270; 549/323

(57) ABSTRACT

This invention provides a polymerizable liquid crystal compound having an α-methylene-γ-butyrolactone site (for example, a polymerizable liquid crystal compound represented by formula (Z2)) useful as a material for optically anisotropic films such as optical compensation films and multidomain films, for example, in polarizing plates and phase difference plates for use in display devices such as liquid crystal display devices, and a polymerizable liquid crystal composition using the same, and their polymer and film.

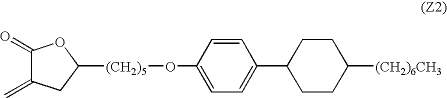

### TECHNICAL FIELD

This invention relates to a liquid crystal compound having polymerizability and liquid crystallinity and a composition including the same, and a polymer obtained by use of them. They have utility in display devices and as a material having optical characteristics such as a recording material or the like, especially as an optical compensation film, such as a polarizing plate and a phase difference plate, for liquid crystal displays.

### BACKGROUND ART

From the standpoint of requirements for improvement in display quality and lightweightness of liquid display devices, there has been an increasing demand for polymer films, in which the molecule orientation structure in the inside thereof is controlled, for use as an optical compensation film. To this end, there has been developed a film of the type which makes use of optical anisotropy of a polymerizable liquid crystal compound. The polymerizable liquid crystal compound used herein is a liquid crystal compound having a polymerizable group and a liquid crystal structural site (i.e. a structural site having a spacer portion and a mesogenic portion), in which an acrylic group is widely used as the polymerizable group. It is usual that the polymer obtained by using such a polymerization liquid crystal compound is obtained by polymerizing a polymerizable liquid crystal compound by irradiation of a radiation ray such as UV light or the like. For instance, there is known a method wherein a specific type of polymerizable liquid crystal compound having an acrylic group is sandwiched between supports and irradiating a radiation ray thereto while maintaining the compound in crystal liquid state (e.g. see Patent Document 1).

It is also known that a photopolymerization initiator is added to a mixture of two types of polymerizable liquid crystal compounds having an acrylic group or a composition of a chiral liquid crystal mixed with such a mixture as mentioned above and irradiating UV light thereto (e.g. see Patent Document 2).

The polymer film obtained in a manner as mentioned above is disposed, as a polarizing plate, a phase difference plate or the like, not only in display devices such as monitors, televisions and the like for indoor use, but also display devices employed in a high temperature environment such as in automobiles. Where a film using a polymerizable liquid crystal compound has a glass transition temperature (hereinafter referred to as Tg) that is lower than a use environment temperature, especially with its use in a high temperature environment, the molecules undergo microscopic fluctuation, with some possibility that the orientation is disturbed, thereby considerably lowering optical anisotropy. It is very important for a display device to keep transparency in a high temperature environment.

Patent Document 1:
JP-A 62-70407

Patent Document 2:
JP-A 9-208957

### DISCLOSURE OF THE INVENTION

### Problems to be Solved by the Invention

The invention has been made under such circumstances as set out above and has for its object the provision of a polymerizable liquid crystal compound capable of yielding a polymer which has optical anisotropy, is able to stably keep transparency under high temperature conditions, and has high Tg with an excellent heat resistance, a polymerizable liquid crystal composition including the same, and a polymer obtained by using them.

### Means for Solving the Problems

We made intensive studies to achieve the above object and, as a result, found that a polymerizable liquid crystal compound having an α-methylene-γ-butyrolactone moiety, which is a polymerizable liquid crystal compound having a lactone ring and a liquid crystal structural moiety, is able to yield a polymer that has optical anisotropy, is able to stably keep transparency at high temperatures, and has high Tg with an excellent heat resistance, thereby arriving at completion of the invention.

More particularly, the invention provides:

1. A polymerizable liquid crystal compound, characterized by represented by the following formula [1]

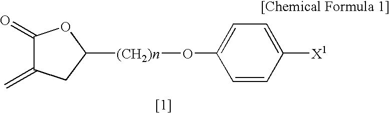

in the formula [1], n is an integer of 2 to 9, and X¹ is of the formula [2] or [3]

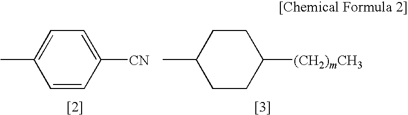

in the formula [3], m is an integer of 4 to 8;
2. A polymerizable liquid crystal composition including at least one type of polymerizable liquid crystal compound of 1;
3. A polymerizable liquid crystal composition including at least one type of polymerizable liquid crystal compound of 1, and other type of polymerizable liquid crystal compound other than the first-mentioned type of polymerizable liquid crystal compound;
4. A polymerizable liquid crystal composition of 3, wherein the other type of polymerizable liquid crystal compound is represented by the following formula [5]

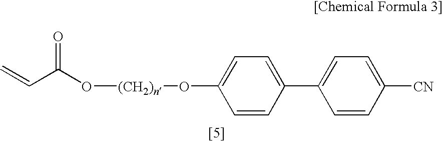

in the formula (5), n′ is an integer of 1 to 6;
5. A polymer obtained by polymerizing the polymerizable liquid crystal compound of 1;
6. A polymer obtained by polymerizing any of the polymerizable liquid crystal compositions of 2 to 4;
7. A film obtained by using the polymerizable liquid crystal compound of 1; and
8. A film obtained by using any of the polymerizable liquid crystal compositions of 2 to 4.

### EFFECTS OF THE INVENTION

The polymerizable liquid crystal compound of the invention yields a polymer having high Tg. The composition including this polymerizable liquid crystal compound also yields a polymer that has optical anisotropy and is able to stably keep transparency in high temperature environments. These polymers are usable as an optically anisotropic film such as a polarizing plate, a phase difference plate or the like. Moreover, the film is useful for application in high temperature environments.

### BEST MODE FOR CARRYING OUT THE INVENTION

The invention is now described in more detail.

### Definitions of Terminology

The meanings of terms used herein are as follows. The term “polymerizable liquid crystal compound” means a compound which has a polymerizable moiety such as an acrylic group, a lactone ring or the like and a liquid crystal structural moiety in the molecule and exhibits a liquid crystal phase. The liquid crystal structure is generally used for indicating liquid crystal molecules and means a structure having a spacer portion and a mesogenic portion. The term “liquid crystal composition” means a composition having a characteristic of exhibiting a liquid crystal phase. The term “liquid crystallinity” means exhibition of a liquid crystal phase.

### [Polymerizable Liquid Crystal Compound]

The polymerizable liquid crystal compound of the invention is represented by the following formula [1] and is a compound having a lactone ring and a liquid crystal structural moiety, particularly, a polymerizable liquid crystal compound having an α-methylene-γ-butyrolactone moiety.

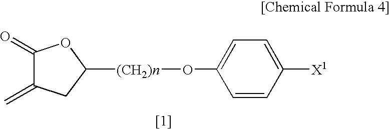

in the formula [1], n is an integer of 2 to 9 and X¹ is of the formula [2] or [3]

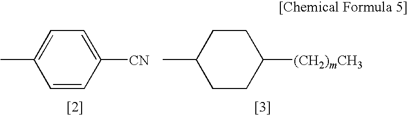

in the formula [3], m is an integer of 4 to 8.

In the compound, the α-methylene-γ-butyrolactone structure is effective in imparting high Tg and a heat resistance to a polymer obtained therefrom.

In the formula [1], recurring units of the methylene group are a moiety that is a so-called spacer portion. In the formula, n represents the number of repetitions of the methylene group and is an integer of 2 to 9. Preferably, n is 2 to 5, more preferably 2 to 4.

In the formula [1], X¹ is a group represented by the formula [2] or [3] and m in the formula [3] is an integer of 4 to 8.

The polymerizable liquid crystal compound represented by the formula [1] shows a liquid crystalline phase such as a smectic phase or a nematic phase. This characteristic is useful in the fields of application using optical anisotropy such as to a polarizing plate, a phase difference plate or the like.

Examples of the polymerizable liquid crystal compound include those compounds of (1) to (18) and the like indicated below although not limited thereto.

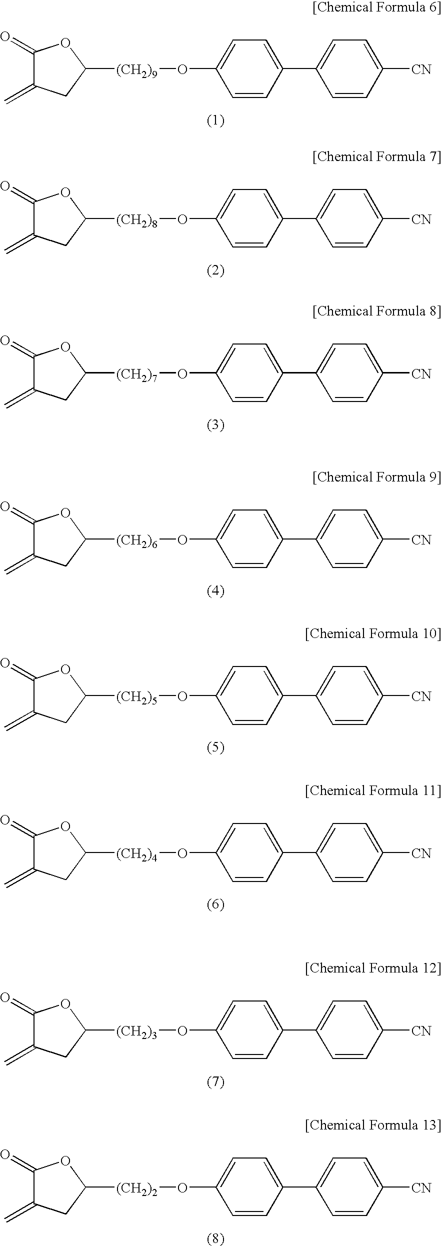

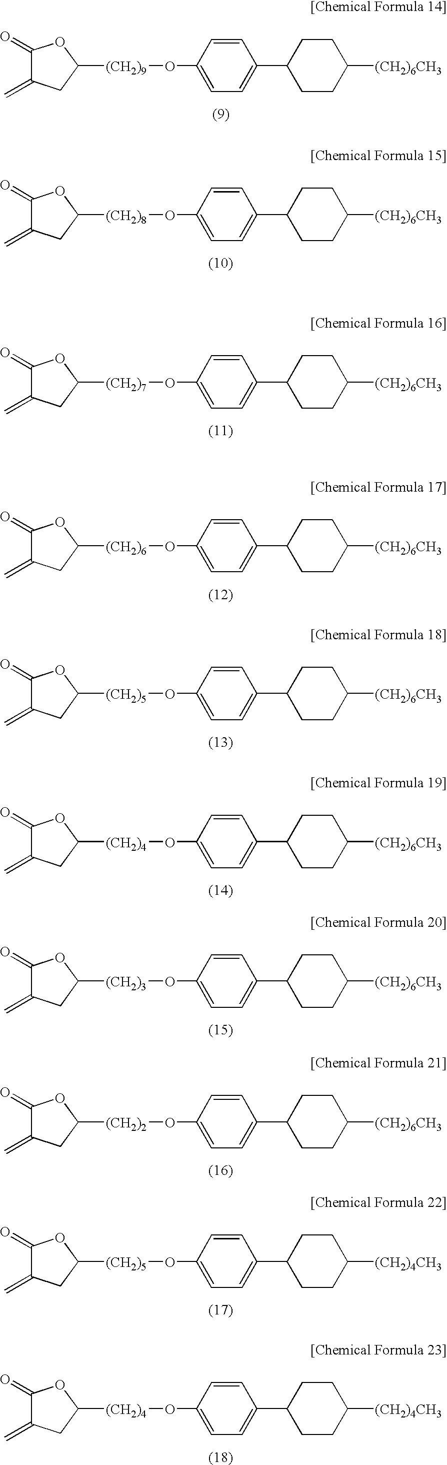

Of these polymerizable liquid crystal compounds, a compound wherein n is an integer of 2 to 5, X¹ is of the formula [3] and m is an integer of 6 is preferred because of the likelihood of obtaining a polymer that is low in temperature at which an isotropic liquid state is attained, exhibits stable crystallinity and has high Tg.

### [Synthesis of Polymerizable Liquid Crystal Compounds]

The polymerizable liquid crystal compound of the invention can be synthesized by combination of techniques in organic synthetic chemistry. Synthetic methods are not limited to specific ones.

For instance, a compound having an α-methylene-γ-butyrolactone structure can be prepared by a technique represented by the following synthetic scheme (A) proposed by Talaga et al (P. Talaga, M. Schaeffer, C. Benezra and J. L. Stampf, Synthesis, 530 (1990)). The technique is a method wherein 2-(bromomethyl)acrylic acid (2-(bromomethyl)propenoic acid) and an aldehyde or ketone are reacted by use of SnCl₂.

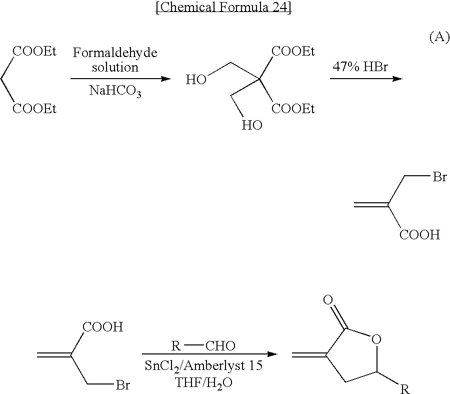

[wherein R represents a monovalent organic group, and Amberlyst (registered trademark) 15 is an ion exchange resin (commercial name, made by Rohm and Haas Co., Inc.)].

In the above synthetic scheme (A), an aldehyde (R—CHO), which is a group represented by the formula [4] in which X¹ is a group represented by the formula (2) or (3), is used as R. More particularly, there can be obtained a polymerizable liquid crystal compound of the invention by use of a procedure of the synthetic scheme (A) using, as a starting material, a compound represented by the formula [4] wherein X¹ is of the formula [2] or [3].

It will be noted that 2-(bromomethyl)acrylic acid (2-(bromomethyl)propenoic acid) can be obtained by a method proposed by Ramarajan et al (K. Ramajaran et al.) [K. Ramarajan, K. Kamalingam. D. J. O'Donnell and K. D. Berlin, Organic Synthesis, vol. 61, 56-59 (1983)].

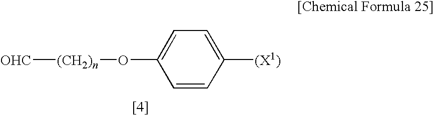

wherein n and X¹, respectively, have the same meanings as defined above.

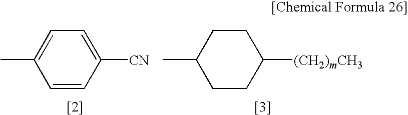

wherein m is an integer of 4 to 8.

The compound represented by the formula [4] may also be obtained by oxidation of a primary alcohol compound. This primary alcohol compound can be obtained by reaction between a bromoalcohol and a phenolic compound. The bromoalcohol and phenolic compound used are, respectively, commercial products and are readily available. The details of these reactions are shown in the following synthetic scheme (B).

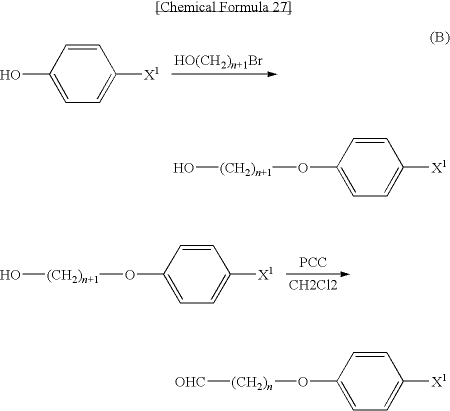

wherein n is an integer of 2 to 9, PCC represents pyridinium chlorochromate, and X¹ has the same meaning as defined before.

Where n=3 and 4, it is preferred to protect the hydroxyl group with tetrahydropyranyl ether beforehand in order to prevent the bromoalcohol compound from undergoing intramolecular cyclization reaction and improve the yield. The details of these reactions are shown in the following synthetic scheme (C).

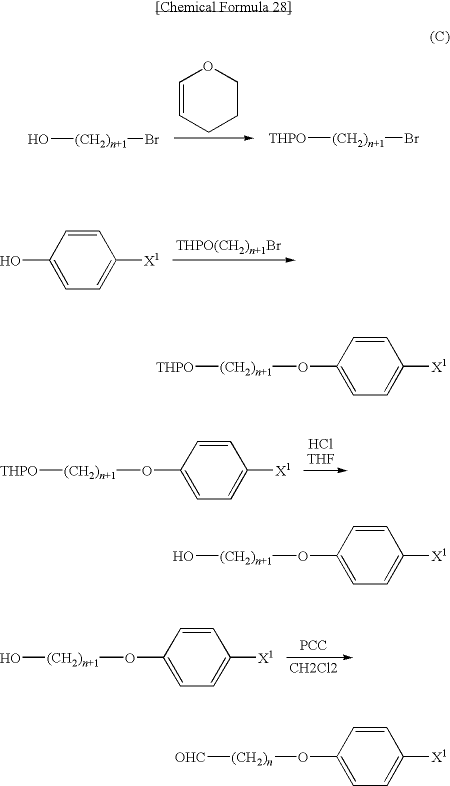

wherein n is an integer of 3 or 4, PCC represents pyridinium chlorochromate, and X¹ has the same meaning as defined above.

### [Polymerizable Liquid Crystal Composition]

The polymerizable liquid crystal composition of the invention contains at least one of polymerization liquid crystal compounds represented by the formula [1]. The polymerizable liquid crystal compounds represented by the formula [1] and contained in the polymerizable liquid crystal composition may be used singly or in admixture of two or more and should preferably exhibit an enantiotropic liquid crystal phase. If two or more are mixed, polymerizable liquid crystal compounds may be appropriately selected and mixed.

So far as the effect of the invention is not impeded, specified compounds indicated below may be admixed with the polymerizable liquid crystal compound of the invention. The specified compounds to be mixed may be used in a plurality thereof. The specified compound may be either of a type showing liquid crystallinity or of a type showing no liquid crystallinity. The specified compounds may have or may not have a polymerizable group such as an acrylic group, a lactone ring or the like. If the specified compound has a polymerizable group, it may be either monofunctional or polyfunctional.

Such specified compounds include compounds that have no polymerizable group but exhibit liquid crystallinity, compounds having no polymerizable group and exhibiting no liquid crystallinity, compounds that have a polymerizable group and exhibit liquid crystallinity but except for those polymerizable liquid crystal compounds of the invention (i.e. other types of polymerizable liquid crystal compounds), and compounds having a polymerizable group and exhibiting no liquid crystallinity. Specific examples include compounds of the formulae (19) to (33) indicated below, nematic liquid crystals, ferrodielectric crystals, polyfunctional acrylate compounds, commercially available liquid crystal compositions, and the like.

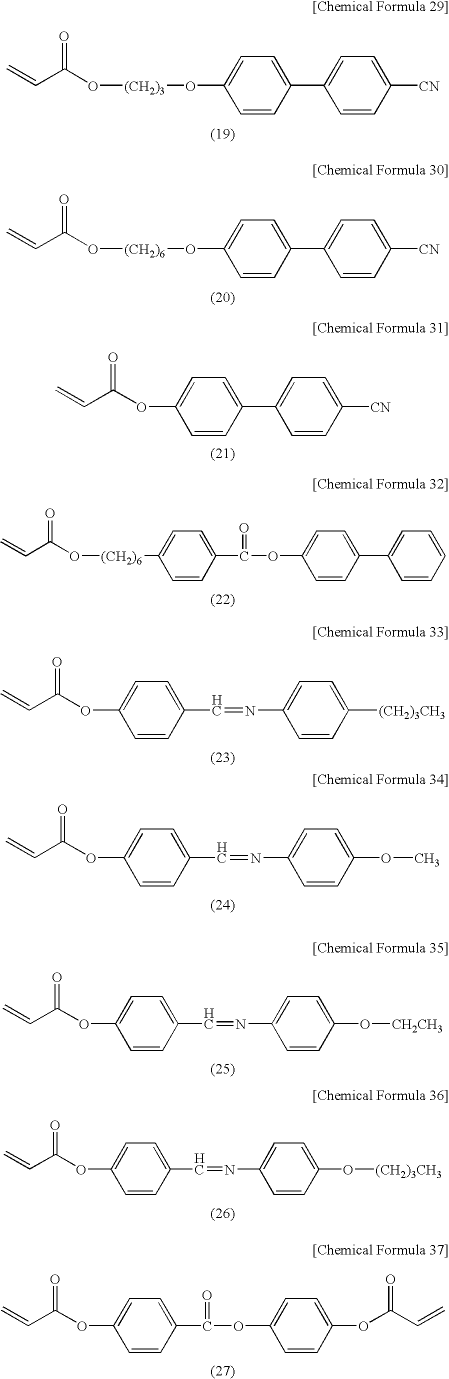

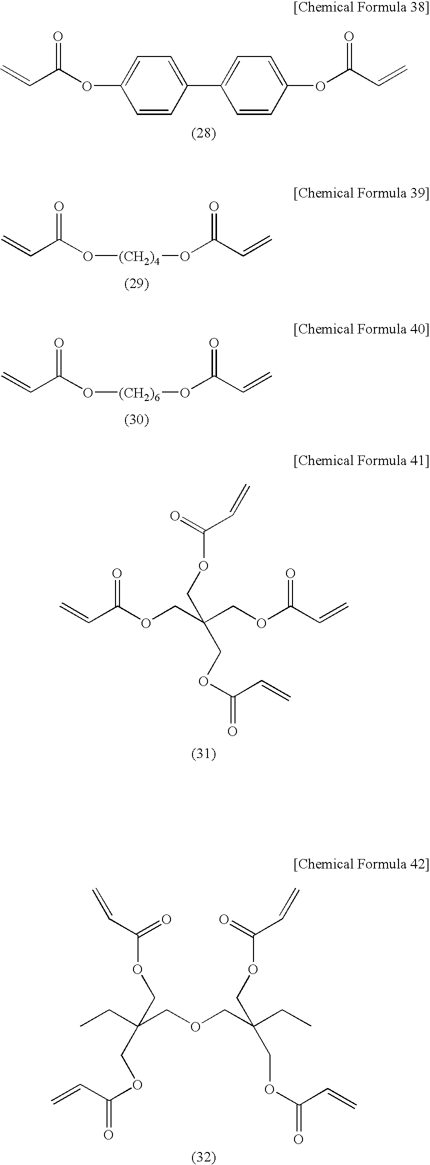

Especially, the compounds of the following general formula [5], which include the compounds of the formulae (20) to (21) and which have a polymerizable group and exhibit liquid crystallinity but except for the polymerizable liquid crystal compounds of the invention (i.e. other types of polymerizable liquid crystal compounds), are favorable.

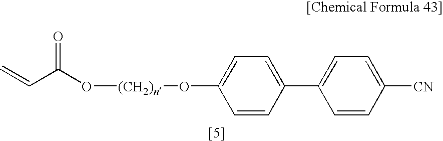

in the formula [5], n′ is an integer of 1 to 6.

As stated above, the specified compounds may be used singly or in combination of two or more. Where a specified compound to be mixed exhibits liquid crystallinity, an amount thereof is preferably not larger than 300 parts by weight of the specified compound relative to 100 parts by weight of a polymerizable liquid crystal compound represented by the formula [1]. Where a specified compound to be mixed exhibits no liquid crystallinity, an amount thereof is preferably not larger than 20 parts by weight, preferably not larger than 10 parts by weight, of the specified compound relative to 100 parts by weight of a polymerizable liquid crystal compound represented by the formula [1].

The polymerizable liquid crystal composition of the invention may be admixed with a photoinitiator, a thermal polymerization initiator, and a sensitizer for the purpose of improving polymerization reactivity.

Examples of the photoinitiator include benzoin ethers such as benzoin methyl ether and the like, benzophenones such as benzophenone and the like, acetophenones such as diethoxyacetophenone and the like, and benzyl ketals such as benzyl dimethyl ketal and the like. The photoinitiators may be used in combination of a plurality thereof and its amount is preferably not larger than 5 parts by weight, more preferably 0.5 to 2 parts by weight, per 100 parts by weight of a total of polymerizable liquid crystal compounds represented by the formula [1] or a total of a polymerizable liquid crystal compound represented by the formula [1] and other type of liquid crystal compound (hereinafter referred to as total polymerizable liquid crystal compounds).

Examples of the thermal polymerization initiators include 2,2-azobisisobutyronitrile and the like. Such thermal polymerization initiators may be used in combination of a plurality thereof and its amount is preferably not larger than 5 parts by weight, preferably 0.5 to 2 parts by weight, per 100 parts by weight of the total polymerizable liquid crystal compounds.

For the photosensitizer, mention is made, for example, of anthracene photosensitizers such as anthracene and the like.

The photosensitizers may be used in combination of a plurality thereof and its amount is preferably not larger than 5 parts by weight per 100 parts by weight of the total polymerizable liquid crystal compounds.

The photoinitiator may be used in combination with at least one of a thermal polymerization initiator and a photosensitizer.

In order to improve the storage stability, a stabilizer may be added to the polymerizable liquid crystal composition of the invention. For the stabilizer, mention is made, for example, of hydroquinones, hydroquinone monoalkyl ethers such as hydroquinone monomethyl ether, 4-t-butylcatechol and the like. These stabilizers may be used in combination of a plurality thereof with its amount being preferably not larger than 0.1 part by weight per 100 parts by weight of the total polymerizable liquid crystal compounds.

The polymerizable liquid crystal composition of the invention may be further admixed with an adhesion promoter for the purpose of improving adhesion to a substrate. Specific examples of the adhesion promoter include chlorosilanes such as trimethylchlorosilane, dimethylvinylchlorosilane, methyldiphenylchlorosilane, chloromethyldimethylchlorosilane and the like, alkoxysilanes such as trimethylmethoxysilane, dimethyldiethoxysilane, methyldimethoxysilane, dimethylvinylethoxysilane, diphenyldimethoxysilane, phenyltriethoxysilane and the like, silazanes such as hexamethyldisilazane, N,N′-bis(trimethylsilyl)urea, dimethyltrimethylsilylamine, trimethylsilylimidazole and the like, silanes such as vinyltrichlorosilane, γ-chloropropyltrimethoxysilane, γ-aminopropyltriethoxysilane, γ-methcryloxypropyltrimethoxysilane, γ-glycidoxypropyltrimethoxysilane, γ-(N-piperidinyl)propyltrimethoxysilane and the like, heterocyclic compounds such as benzotriazole, benzimidazole, indazole, imidazole, 2-mercaptobenzimidazole, 2-mercaptobenzothiazole, 2-mercaptobenzooxazole, urazole, thiouracil, mercaptoimidazole, mercaptopyrimidine and the like, and urea compounds or thiourea compounds such as 1,1-dimethylurea, 1,3-dimethylurea and the like.

These adhesion promoters may be used in combination of a plurality thereof and its amount is preferably not larger than 1 part by weight per 100 parts by weight of the total polymerizable liquid crystal compounds.

Further, the polymerizable liquid crystal composition of the invention may be admixed with an organic solvent for the purpose of viscosity adjustment. In this case, the composition may not exhibit any crystallinity in a condition of containing an organic solvent.

The organic solvents include, for example, ethers such as tetrahydrofuran, dioxane and the like, aromatic hydrocarbons such as benzene, toluene, xylene and the like, polar solvents such as N,N-dimethylformamide, N-methyl-2-pyrrolidone and the like, esters such as ethyl acetate, butyl acetate, ethyl lactate and the like, alkoxyesters such as methyl 3-methoxypropionate, methyl 2-methoxypropionate, ethyl 3-methoxypropionate, ethyl 2-methoxypropionate, ethyl 3-ethoxypropionate, ethyl 2-ethoxypropionate and the like, glycol dialkyl ethers such as ethylene glycol dimethyl ether, propylene glycol dimethyl ether and the like, diglycol dialkyl ethers such as diethylene glycol dimethyl ether, diethylene glycol diethyl ether, diethylene glycol methyl ethyl ether dipropylene glycol dimethyl ether and the like, glycol monoalkyl ethers such as ethylene glycol monomethyl ether, ethylene glycol monoethyl ether, propylene glycol monomethyl ether, propylene glycol monoethyl ether and the like, diglycol monoalkyl ethers such as diethylene glycol monomethyl ether, diethylene glycol monoethyl ether, dipropylene glycol monomethyl ether, dipropylene glycol monoethyl ether and the like, glycol monoalkyl ether esters such as propylene glycol monomethyl ether acetate, carbitol acetate, ethyl cellosolve acetate and the like, and ketones such as cyclohexanone, methyl ethyl ketone, methyl isobutyl ketone, 2-heptanone and the like.

These organic solvents may be used singly or in combination of two or more. Of these, from the standpoint of the safety on earth's and working environments, propylene glycol monomethyl ether, propylene glycol monomethyl ether acetate, ethyl lactate and cyclohexanone are preferred.

For the purpose of improving affinity for a substrate, the polymerizable liquid crystal composition of the invention may contain a surfactant. Although such a surfactant may be a fluorine-based surfactant, a silicone-based surfactant, nonionic surfactant or the like and is not limited to any specific type, a fluorine-based surfactant having an improving effect of affinity for a substrate is preferred.

Specific examples of the fluorine-based surfactant (hereinafter indicated as commercial names) F TOP EF301, EF303 or EF352 (Tochem Products Co., Ltd.), Megafacks F171, F173 or R-30 (Dainippon Ink and Chemicals, Incorporated), Florard FC430 or FC 431 (Sumitomo 3M Co., Ltd.), Asahi Guard ASG710, Surflon S-382, SC101, SC102, SC103, SC104, SC105 or SC106 (made by Asahi Glass Co., Ltd.) although not limitative thereto. The surfactants may be used in combination of a plurality thereof.

Examples of a preferred polymerizable liquid crystal composition include a liquid crystal composition including 100 parts by weight of a polymerizable liquid crystal compound represented by the formula [1] and not larger than 5 parts by weight of a photoinitiator, a liquid crystal composition including 100 parts by weight of a polymerizable liquid crystal compound represented by the formula [1], not larger than 20 parts by weight of a specified compound not exhibiting crystallinity and not larger than 5 parts by weight of a photoinitiator, and a polymerizable liquid crystal composition including 100 parts by weight of a polymerizable liquid crystal compound represented by the formula [1], not larger than 300 parts by weight of a specified compound exhibiting crystallinity and not larger than 5 parts by weight of a photoinitiator.

The manner of obtaining the polymerizable liquid crystal composition of the invention is not critical. The ingredients of the polymerizable liquid crystal composition may be mixed at one time, or may be successively mixed together. If one ingredient is made of plural compounds, the plural compounds mixed beforehand may be mixed with other ingredients, or all the components may be individually, successively mixed.

It is preferred that the polymerizable liquid crystal composition of the invention exhibits an enantiotropic liquid crystal phase at room temperature in order that upon manufacture of an optically anisotropic body, unintended thermal polymerization is prevented from being induced in the course of photopolymerization in a liquid state and it is made easy to fix the molecules in a uniformly oriented state. Moreover, it is preferred that if the polymerizable liquid crystal composition contains an organic solvent, an enantiotropic liquid crystal phase is shown at room temperature upon removal of the solvent.

### [Polymers and Films]

The polymerizable liquid crystal compound or polymerizable liquid crystal composition of the invention is converted to a polymer upon irradiation of light or by application of heat. It will be noted that where a polymer is obtained from a polymerizable liquid crystal compound or a polymerizable liquid crystal composition of the invention, such a photoinitiator, thermal polymerization initiator, sensitizer and the like as set out with respect to the polymerizable liquid crystal composition can be used in amounts defined before, respectively. With a polymerizable liquid crystal compound, the amounts, which correspond to those amounts relative to 100 parts by weight of a polymerizable liquid crystal compound taken in place of 100 parts by weight of the total polymerizable liquid crystal compounds, may be used, respectively.

For a method of obtaining a film, mention is made of a method wherein a polymerizable liquid crystal compound or a polymerizable liquid crystal composition is sandwiched between two substrates and light is irradiated thereon for polymerization, and a method wherein a polymerizable liquid crystal compound or a polymerizable liquid crystal composition is applied onto a substrate by a spin-coating, casting or the like method, followed by irradiation of light.

As a substrate, there can be used glass, quartz, plastic sheets, color filters, plastic films such as triacetylcellulose (TAC), and the like. Alternatively, there may also be used glass, plastic sheets, plastic films and the like substrates wherein a functional thin film such as of ITO or the like has been formed on one side thereof, and belts and drums plated or vacuum deposited with a metal such as a stainless steel, chromium or aluminium. For the purpose of improving the orientation of a film, it is preferred to subject a substrate to orientation treatment. For an orientation treatment, mentions is made of ordinarily known methods including a method wherein an orientation material containing a polyimide precursor, a polyimide, polyvinyl cinnamate or the like is coated, followed by orientation treatment by rubbing or irradiation of polarized UV light, a method of forming an obliquely vacuum-deposited film of silicon dioxide, and a method of forming a Langmuir film.

In a method of sandwiching a polymerizable liquid crystal compound or polymerizable liquid crystal composition between two substrates, a cell of two substrates between which a space is formed such as by use of a spacer is provided and a polymerizable liquid crystal compound or polymerizable liquid crystal composition is charged into the cell by use of a capillary phenomenon or by a method of reducing the pressure in the space of the cell, followed by polymerization by light irradiation.

For a simpler procedure, there is mentioned a method wherein a polymerizable liquid crystal compound or polymerizable liquid crystal composition is placed on a substrate provided with a spacer or the like and the other substrate is superposed to provide a cell, followed by polymerization by irradiation of light. In this case, the polymerizable liquid crystal compound or polymerizable liquid crystal composition may be used after being fluidized or may be fluidized by application of heat or the like after placement on a substrate. In this connection, however, it is necessary that the polymerizable liquid crystal compound or polymerizable liquid crystal composition be fluidized prior to superposition of the other substrate.

In a method of coating a polymerizable liquid crystal compound or polymerizable liquid crystal composition, a step of heating such as by a hot plate or the like may be added, if necessary, on the midway between a step of coating the compound or liquid crystal composition and a step of polymerization by application of light or heat. Especially, in case where there is used a polymerizable liquid crystal compound or polymerizable liquid crystal composition containing an organic solvent therein, this step is effective because the organic solvent can be removed.

In any of the above methods, an oriented film having optical anisotropy can be obtained by polymerizing a polymerizable liquid crystal compound or polymerizable liquid crystal composition under conditions sufficient to give a liquid crystal phase.

In order to obtain a polymer in a multi-domain wherein orientations differ from each other in every adjacent domain, there is used a multi-domaining method in a polymerization step or a multi-domaining method of the orientation treatment of a substrate.

The multi-domaining method in a polymerization step includes a method wherein a polymerizable liquid crystal compound or polymerizable liquid crystal composition in a liquid crystal state is exposed to UV light through a mask to form a polymerized domain and the other domain is polymerized in an isotropic liquid state.

The method of multi-domaining the orientation treatment of a substrate includes a method of rubbing an orientation material formed on the substrate through a mask or a method of irradiating UV light through a mask.

According to these methods, there is obtained a multi-domained substrate wherein the rubbed domain or UV-irradiated domain is an oriented portion, and the other is a non-treated portion. A polymerizable liquid crystal compound or polymerizable liquid crystal composition formed on this multi-domained substrate suffers an influence of the orientation material layer and is multi-domained. Aside from such orientation treatment methods, a method of utilizing an electric field or a magnetic filed may also be used.

The polymerizable liquid crystal compound and the polymerizable liquid crystal composition using the compound according to the invention can be utilized for the formation of a film having optical anisotropy and can be conveniently used as a polarizing plate, a phase difference plate and the like. This film is good at transparency at high temperatures and can be beneficially used as electronic devices employed in high temperature environments such as on-vehicle display apparatus.

### EXAMPLES

Examples and comparative examples are now described to more particularly illustrate the invention, which should not be construed as limiting to the following examples. It will be noted that measuring methods used in examples are as illustrated below.

### [1] Measurement of NMR

A compound was dissolved in deuterated chloroform (CDCl₃), followed by measurement with ¹H-NMR of 300 MHz (made by JEOL Ltd.). In Example 3, however, deuterated dimethylsulfoxide (DMSO-d6) was used in place of deuterated chloroform.

### [2] IR Measurement

A compound was shaped into a pellet by use of potassium bromide, followed by measurement by use of NEXUS670FT-IR (made by Nicolet).

### [3] Measurement of MS(FAB+)

The mass spectra of a compound was measured by use of JEOL LX-1000 made by JEOL ltd.

### [4] Observation of a Liquid Crystal Phase

Identification of a liquid crystal phase was carried out by heating a sample on a hot stage (MATS-2002S, made by Tokai Hit Co., Ltd.) and observing by use of a polarization microscope (made by Nikon Corporation). The phase transition temperature was measured by use of a differential scanning thermal analysis apparatus (DSC3100SR)(hereinafter referred to as DSC) made by Mac Science Co., Ltd., under conditions of a scan rate of 10° C./minute. (In this connection, however, in Example 6, the measurement was made at 5° C./minute.)

### [5] Measurement of a Molecular Weight

The molecular weight was measurement by dissolving a sample in tetrahydrofuran (hereinafter referred to as THF) at a rate of 0.2 wt %, followed by measurement by use of room temperature gel permeation chromatography (hereinafter referred to as GPC), made by JASCO. For the measurement, a column (Shodex GPC KF-803L), made by Showa Denko K. K., was used. By the measurement, there were obtained a number average molecular weight and a weight average molecular weight, calculated as polystyrene.

In this regard, however, in Example 9, a sample was dissolved in dimethylformamide (hereinafter referred to as DMF) at a rate of 0.2 wt %, followed by measurement by use of GPC (Shodex GPC 101) made by Showa Denko K. K., and a column (Shodex KD-803, KD-805) made by Showa Denko K. K., thereby obtaining a number average molecular weight and a weight average molecular weight, calculated as polyethylene glycol.

### [6] Measurement of a Glass Transition Temperature

The glass transition temperature (Tg) was measured by use of a differential scanning thermal analysis apparatus (DSC3100SR) made by Mac Science Co., Ltd. under conditions of a scan rate of 10° C./minute.

### [7] 5% Weight Loss Temperature

The thermogravimetric analysis apparatus (TG-DTA2000-SR), made by Mac Science Co., Ltd., was used for the measurement and a temperature at which the weight loss of a sample became 5% was determined as a 5% weight loss temperature.

### [8] Measurement of Transmittance

Using Spectral Haze Meter (TC-1800H) made by Tokyo Denshoku Co., Ltd., a transmittance at a wavelength of 550 nm was measured.

### [9] Retardation Value of a Film

The retardation value of a film was measured by use of a retardation measuring device (ORC Co., Ltd.). In Examples 17, 18 19 and Comparative Example 3, a retardation measuring device (RETS-100, made by Otsuka Electronic Co., Ltd.) was used for the measurement.

### Example 1

### Synthesis of Polymerizable Liquid Crystal Compound (Z1)

First, 5.0 g (25.6 mmols) of 4-cyano-4′-hydroxybiphenil, 4.6 g (25.6 mmols) of 6-bromo-1-hexanol, 7.0 g (50 mmols) of potassium carbonate and 50 ml of acetone were added to a 100 ml egg plant-type flask equipped with a condenser tube to provide a mixture, followed by reaction under stirring at 64° C. for 24 hours. After completion of the reaction, the solvent was distilled off under reduced pressure to obtain a yellow wet solid. Thereafter, this solid and 70 ml of water were mixed, to which 50 ml of diethyl ether was added for extraction. The extraction was repeated three times.

The separated organic phase was dried over anhydrous magnesium sulfate, followed by filtration and distilling off the solvent under reduced pressure to obtain a yellow solid. This solid was dissolved in 3 ml of ethyl acetate, followed by purification with column chromatography (column: silica gel 60 0.063 to 0.200 mm, made by Merck Ltd., eluate: hexane/ethyl acetate=1/1). The solvent was distilled off from the thus obtained solution to obtain 6.9 g of a white solid. The results of measurement of the solid with NMR are shown below. From the results, it was confirmed that this white solid was an intermediate compound (P1) shown in the following synthetic scheme (i). (Yield: 91%)

¹H-NMR (DMSO-d6) δ: 1.26 (m, 6H), 1.69 (m, 2H), 3.37 (t, 2H), 4.03 (t, 2H), 7.06 (d, 2H), 7.69 (d, 2H), 7.85 (m, 4H).

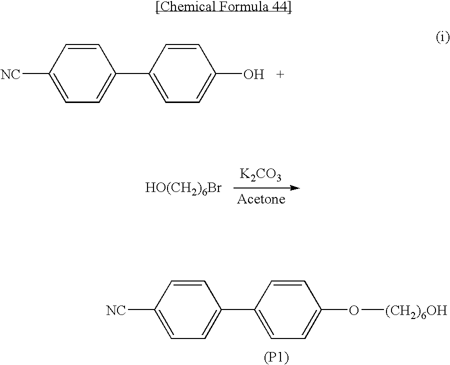

Next, 2.2 g (10.0 mmols) of pyridinium chlorochromate (hereinafter referred to as PCC) and 30.0 ml of CH₂Cl₂ were placed in a 200 ml three-necked flask equipped with a condenser tube and mixed with stirring, under which a solution of 2.95 g (10.0 mmols) of the intermediate compound (P1) obtained in the same manner as set out hereinabove dissolved in CH₂Cl₂ (50.0 ml) was dropped, followed by further stirring at 40° C. for 0.5 hour. Subsequently, 90 ml of diethyl ether was added to the solution except for an oily matter attached to the walls of the flask and filtered under reduced pressure, followed by distilling off the solvent under reduced pressure to obtain a dark green, wet solid.

This solid was dissolved in 3 ml of ethyl acetate and purified with silica column chromatography (column: silica gel 60 0.063 to 0.200 mm, made by Merck Ltd., eluate: hexane/ethyl acetate=1/1). The solvent was removed from the resulting solution to obtain 2.8 g of a colorless solid. The results of measurement of this colorless solid with NMR are shown below. From these results, it was confirmed that this colorless solid was an intermediate compound (Q1) shown in the following synthetic scheme. (Yield: 93%)

¹H-NMR (CDCl₃) δ: 1.84 (m, 6H), 2.50 (m, 2H), 4.02 (m, 2H), 6.99 (d, 2H), 7.53 (d, 2H), 7.91 (m, 4H), 9.80 (s, 1H).

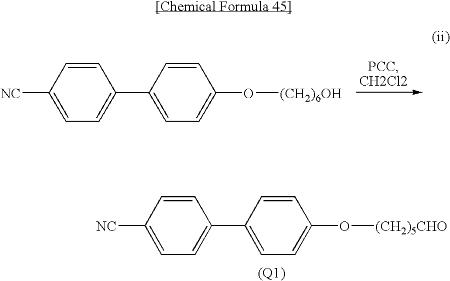

Finally, 3.0 g (10.0 mmols) of the intermediate compound (Q1) obtained in the same manner as set out above, 1.65 g (10.0 mmols) of 2-(bromomethyl)acrylic acid, 1.6 g of Amberlyst (registered trade mark) 15 (commercial name of Rohm and Haas Co., Inc.), 16.0 ml of THF, 1.9 g (10.0 mmols) of tin (II) chloride and 4.0 ml of pure water were added to a 50 ml egg plant-type flask equipped with a condenser tube to provide a mixture, followed by reaction under stirring at 70° C. for 7 hours. After completion of the reaction, the reaction solution was filtered under reduced pressure and mixed with 30 ml of pure water, to which 50 ml of diethyl ether was added for extraction. The extraction was repeated three times.

The organic phase obtained after the extraction was dried over anhydrous magnesium sulfate, followed by filtration under reduced pressure and distilling off the solvent from the solution to obtain a yellow solid. This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 1.5 g of a white solid. This solid was subjected to measurements of NMR, IR and MS, revealing that the white solid consisted of an intended polymerizable liquid crystal compound (Z1) shown in the following synthetic scheme (iii). (Yield: 41%)

The results of the measurements are shown below.

¹H-NMR (CDCl₃) δ: 1.57 (m, 6H), 1.85 (m, 2H), 2.60 (m, 1H), 3.05 (m, 1H), 4.01 (t, 2H), 4.54 (m, 1H), 5.63 (m, 1H), 6.23 (m, 1H), 7.00 (d, 2H), 7.52 (d, 2H), 7.68 (m, 4H).

IR (KBr, cm⁻¹): 2934, 2228, 1761, 1664.

MS(FAB+): 361(M+)

As a result of observation of the liquid crystallinity of this polymerizable liquid crystal compound (Z1), it was found that the compound converted to an isotropic liquid state at 84° C. and underwent phase transition into a liquid crystal phase (nematic phase) at 61° C. during the temperature drop.

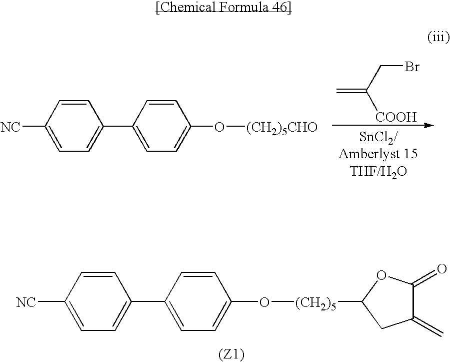

### Example 2

### Synthesis of Polymerizable Liquid Crystal Compound (Z2)

First, 20.0 g (72.9 mmols) of p-(trans-4-heptylcyclo-hexyl)phenol, 13.2 g (72.9 mmols) of 6-bromo-1-hexanol, 23.2 g (160 mmols) of potassium carbonate and 250 ml of acetone were added to a 500 ml egg plant-type flask equipped with a condenser tube to provide a mixture, followed by reaction under stirring at 64° C. for 48 hours. After completion of the reaction, the solvent was distilled off under reduced pressure to obtain a light brown, wet solid. Thereafter, this solid and 150 ml of water were mixed, to which 80 ml of diethyl ether was added for extraction. The extraction was repeated three times.

The separated organic phase was dried over anhydrous magnesium sulfate, followed by filtration and distilling off the solvent under reduced pressure to obtain a light brown, wet solid. This solid was dissolved in 8 ml of ethyl acetate, followed by purification with column chromatography (column: silica gel 60 0.063 to 0.200 mm, made by Merck Ltd., eluate: hexane/ethyl acetate=2/1). The solvent was distilled off from the thus obtained solution to obtain 24.3 g of a light milky-white solid. The results of measurement of the solid with NMR are shown below. From the results, it was confirmed that this light milky-white solid was an intermediate compound (P2) shown in the following synthetic scheme (iv). (Yield: 89%)

¹H-NMR (CDCl₃) δ: 0.89 (t, 3H), 1.10 (m, 2H), 1.26 to 1.65 (m, 21H), 1.83 (m, 6H), 2.40 (m, 1H), 3.63 (t, 2H), 3.93 (t, 2H), 6.81 (d, 2H), 7.11 (d, 2H).

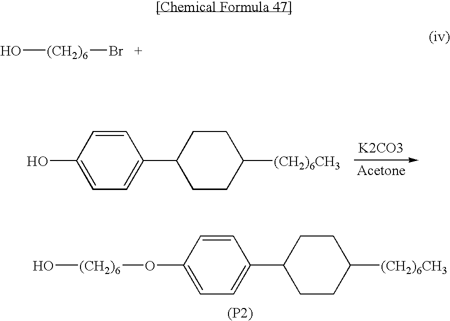

Next, 2.2 g (10.0 mmols) of PCC and 30.0 ml of CH₂Cl₂ were placed in a 200 ml three-necked flask equipped with a condenser tube and mixed with stirring, under which a solution of 3.8 g (10.0 mmols) of the intermediate compound (P2) obtained in the same manner as set out hereinabove dissolved in CH₂Cl₂ (30.0 ml) was dropped, followed by further stirring at 40° C. for 0.5 hour. Subsequently, 90 ml of diethyl ether was added to the solution except for an oily matter attached to the walls of the flask and filtered under reduced pressure, followed by distilling off the solvent under reduced pressure to obtain a dark green, wet solid.

This solid was dissolved in 3 ml of ethyl acetate and purified with silica column chromatography (column: silica gel 60 0.063 to 0.200 mm, made by Merck Ltd., eluate: hexane/ethyl acetate=2/1). The solvent was removed from the resulting solution to obtain 3.0 g of a light milky-white solid. The results of measurement of this light milky-white solid with NMR are shown below. From these results, it was confirmed that this light milky-white solid was an intermediate compound (Q2) shown in the following synthetic scheme (v). (Yield: 80%)

¹H-NMR (CDCl₃) δ: 0.89 (t, 3H), 1.05 (m, 2H), 1.20 to 1.65 (m, 19H), 1.83 (m, 6H), 2.40 (m, 1H), 2.45 (t, 2H), 3.93 (t, 2H), 6.80 (d, 2H), 7.10 (d, 2H), 9.78 (s, 1H).

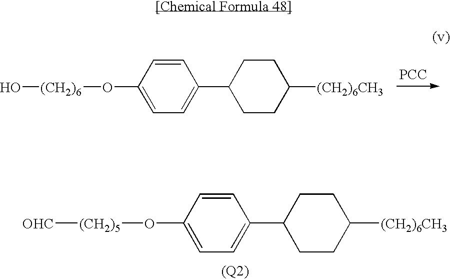

Finally, 1.9 g (5.0 mmols) of the intermediate compound (Q2) obtained in the same manner as set out above, 0.8 g (5.0 mmols) of 2-(bromomethyl)acrylate, 0.8 g of Amberlyst (registered trade mark) 15 (commercial name of Rohm and Haas Co., Inc.), 8.0 ml of THF, 0.95 g (5.0 mmols) and 2.0 ml of pure water were added to a 50 ml egg plant-type flask equipped with a condenser tube to provide a mixture, followed by reaction under stirring at 70° C. for 24 hours. After completion of the reaction, the reaction solution was filtered under reduced pressure and mixed with 30 ml of pure water, to which 30 ml of diethyl ether was added for extraction. The extraction was repeated three times. The organic phase obtained after the extraction was dried over anhydrous magnesium sulfate, followed by filtration under reduced pressure and distilling off the solvent from the solution to obtain a light brown solid.

This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 0.94 g of a white solid. This solid was subjected to measurements of NMR, IR and MS, revealing that the white solid consisted of an intended polymerizable liquid crystal compound (Z2) shown in the following synthetic scheme (vi). (Yield: 43%)

The results of the measurements are shown below.

¹H-NMR (CDCl₃) δ: 0.87 (t, 3H), 1.05 (m, 2H), 1.20 to 1.90 (m, 21H), 1.84 (m, 6H), 2.40 (m, 1H), 2.60 (m, 1H), 3.04 (m, 1H), 3.93 (t, 2H), 4.52 (m, 1H), 5.61 (m, 1H), 6.23 (m, 1H), 6.81 (d, 2H), 7.12 (d, 2H).

IR (KBr, cm⁻¹): 2921, 1758, 1672.

MS(FAB+): 440(M+)

As a result of observation of the liquid crystallinity of this polymerizable liquid crystal compound (Z2), it was found that the compound converted to an isotropic liquid state at 74° C. and underwent phase transition into a smectic A phase at 65° C. and a smectic X phase (not determined smectic phase) at 58° C. during the temperature drop.

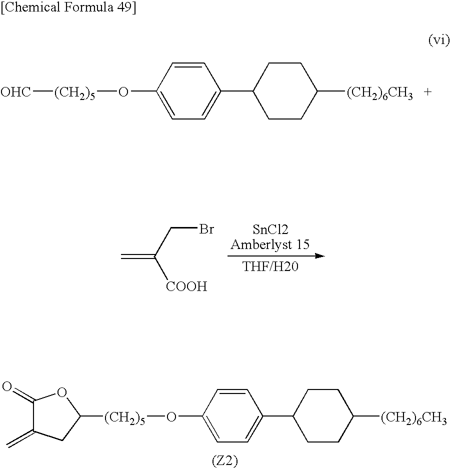

### Example 3

### Synthesis of Polymerizable Liquid Crystal Compound (Z3)

First, 5.0 g (25.6 mmols) of 4-cyano-4′-hydroxybiphenil, 6.1 g (25.6 mmols) of 10-bromo-1-decanol, 7.0 g (50 mmols) of potassium carbonate and 50 ml of acetone were added to a 100 ml egg plant-type flask equipped with a condenser tube to provide a mixture, followed by reaction under stirring at 64° C. for 48 hours. After completion of the reaction, the solvent was distilled off under reduced pressure to obtain a yellow wet solid. Thereafter, this solid and 70 ml of water were mixed, to which 50 ml of diethyl ether was added for extraction. The extraction was repeated three times.

The separated organic phase was dried over anhydrous magnesium sulfate, followed by filtration and distilling off the solvent under reduced pressure to obtain a yellow solid. This solid was dissolved in 3 ml of ethyl acetate, followed by purification with column chromatography (column: silica gel 60 0.063 to 0.200 mm, made by Merck Ltd., eluate: hexane/ethyl acetate=1/1). The solvent was distilled off from the thus obtained solution to obtain 8.2 g of a white solid. The results of measurement of the solid with NMR are shown below. From the results, it was confirmed that this white solid was an intermediate compound (P3) shown in the following synthetic scheme (vii). (Yield: 91%)

¹H-NMR (DMSO-d6) δ: 1.26 (m, 14H), 1.69 (m, 2H), 3.37 (t, 2H), 4.03 (t, 2H), 7.06 (d, 2H), 7.69 (d, 2H), 7.85 (m, 4H).

Next, 1.08 g (5.0 mmols) of PCC and 15.0 ml of CH₂Cl₂ were placed in a 200 ml three-necked flask equipped with a condenser tube and mixed with stirring, under which a solution of 1.76 g (5.0 mmols) of the intermediate compound (P3) obtained in the same manner as set out hereinabove dissolved in CH₂Cl₂ (50.0 ml) was dropped, followed by further stirring at 40° C. for 0.5 hour. Subsequently, 90 ml of diethyl ether was added to the solution except for an oily matter attached to the walls of the flask and filtered under reduced pressure, followed by distilling off the solvent under reduced pressure to obtain a dark green, wet solid.

This solid was dissolved in 3 ml of ethyl acetate and purified with silica column chromatography (column: silica gel 60 0.063 to 0.200 mm, made by Merck Ltd., eluate: hexane/ethyl acetate=1/1). The solvent was distilled off from the resulting solution to obtain 1.5 g of a colorless solid. The results of measurement of this colorless solid with NMR are shown below. From these results, it was confirmed that this colorless solid was an intermediate compound (Q3) shown in the following synthetic scheme (vii). (Yield: 84%)

¹H-NMR (CDCl₃) δ: 1.47 (m, 10H), 1.64 (m, 2H), 1.83 (m, 2H), 2.43 (m, 2H), 4.00 (m, 2H), 6.99 (d, 2H), 7.53 (d, 2H), 7.67 (m, 4H), 9.76 (s, 1H).

Finally, 1.1 g (3.0 mmols) of the intermediate compound (Q3) obtained in the same manner as set out above, 0.50 g (3.0 mmols) of 2-(bromomethyl)acrylic acid, 0.5 g of Amberlyst (registered trade mark) 15 (commercial name of Rohm and Haas Co., Inc.), 5.3 ml of THF, 0.60 g (3.0 mmols) of tin (II) chloride and 1.3 ml of pure water were added to a 50 ml egg plant-type flask equipped with a condenser tube to provide a mixture, followed by reaction under stirring at 70° C. for 6 hours. After completion of the reaction, the reaction solution was filtered under reduced pressure and mixed with 20 ml of pure water, to which 50 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a yellow solid.

This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 0.79 g of a white solid. This solid was subjected to measurements of NMR, IR and MS, revealing that the white solid consisted of an intended polymerizable liquid crystal compound (Z3) shown in the following synthetic scheme (vii). (Yield: 63%)

The results of the measurements are shown below.

¹H-NMR (CDCl₃) δ: 1.33 (m, 12H), 1.47 (m, 2H), 1.81 (m, 2H), 2.60 (m, 1H), 3.06 (m, 1H), 4.00 (t, 2H), 4.52 (m, 1H), 5.62 (m, 1H), 6.22 (m, 1H), 7.00 (d, 2H), 7.51 (d, 2H), 7.66 (m, 4H).

IR(KBr, cm⁻¹): 2934, 2231, 1750, 1664.

MS(FAB+): 418(M+)

As a result of observation of the liquid crystal phase of this polymerizable liquid crystal compound (Z3), it was found that the compound converted to an isotropic liquid state at 79° C. and underwent phase transition into a nematic phase at 62° C. during the temperature drop.

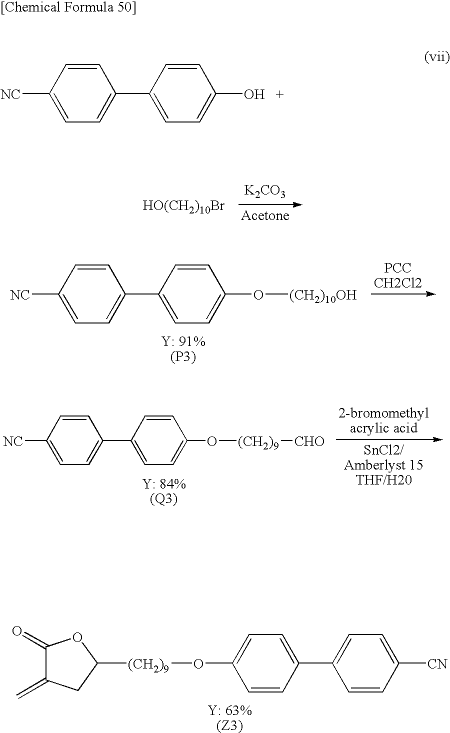

### Example 4

### Synthesis of Polymerizable Liquid Crystal Compound (Z4)

First, 9.8 g (50.0 mmols) of 4-cyano-4′-hydroxybiphenil, 7.0 g (50.0 mmols) of 3-bromo-1-propanol, 13.8 g (100 mmols) of potassium carbonate and 150 ml of acetone were added to a 500 ml egg plant-type flask equipped with a condenser tube to provide a mixture, followed by reaction under stirring at 64° C. for 48 hours. After completion of the reaction, the solvent was distilled off under reduced pressure to obtain a yellow wet solid. Thereafter, this solid and 140 ml of water were mixed, to which 100 ml of diethyl ether was added for extraction. The extraction was repeated three times. The separated organic phase was dried over anhydrous magnesium sulfate, followed by filtration and distilling off the solvent under reduced pressure to obtain a yellow solid.

This solid was purified by recrystallization with use of hexane/ethyl acetate=2/1. 8.7 g of a white solid was obtained. The results of measurement of the solid with NMR are shown below. From the results, it was confirmed that this white solid was an intermediate compound (P4) shown in the following synthetic scheme (vii). (Yield: 70%)

¹H-NMR (CDCl₃) δ: 2.09 (m, 2H), 3.90 (t, 2H), 4.20 (t, 2H), 6.99 (d, 2H), 7.52 (d, 2H), 7.66 (m, 4H).

Next, 4.32 g (20.0 mmols) of PCC and 50.0 ml of CH₂Cl₂ were placed in a 200 ml three-necked flask equipped with a condenser tube and mixed with stirring, under which a solution of 5.06 g (20.0 mmols) of the intermediate compound (P4) obtained in the same manner as set out hereinabove dissolved in CH₂Cl₂ (50.0 ml) was dropped, followed by further stirring at 40° C. for 0.5 hour. Subsequently, 100 ml of diethyl ether was added to the solution except for an oily matter attached to the walls of the flask and filtered under reduced pressure, followed by distilling off the solvent under reduced pressure to obtain a dark green, wet solid.

This solid was dissolved in 3 ml of ethyl acetate and purified with silica column chromatography (column: silica gel 60 0.063 to 0.200 mm, made by Merck Ltd., eluate: hexane/ethyl acetate=1/1). The solvent was distilled off from the resulting solution to obtain 3.2 g of a colorless solid. The results of measurement of this solid with NMR are shown below. From these results, it was confirmed that this colorless solid was an intermediate compound (Q4) shown in the following synthetic scheme (viii). (Yield: 64%)

¹H-NMR (CDCl₃) δ: 2.98 (t, 2H), 4.39 (t, 2H), 6.99 (d, 2H), 7.55 (d, 2H), 7.69 (m, 4H), 9.90 (s, 1H).

Finally, 2.5 g (10.0 mmols) of the intermediate compound (Q4) obtained in the same manner as set out above, 1.65 g (10.0 mmols) of 2-(bromomethyl)acrylic acid, 1.6 g of Amberlyst (registered trade mark) 15 (commercial name of Rohm and Haas Co., Inc.), 16.0 ml of THF, 1.9 g (10.0 mmols) of tin (II) chloride and 4.0 ml of pure water were added to a 50 ml egg plant-type flask equipped with a condenser tube to provide a mixture, followed by reaction under stirring at 70° C. for 24 hours. After completion of the reaction, the reaction solution was filtered under reduced pressure and mixed with 30 ml of pure water, to which 50 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a yellow solid.

This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 0.8 g of a white solid. The results of measurement of the solid with NMR are shown below. From the results, it was confirmed that the white solid consisted of an intended polymerizable liquid crystal compound (Z4) shown in the following synthetic scheme (viii). (Yield: 25%)

¹H-NMR (CDCl₃) δ: 2.18 (m, 2H), 2.76 (m, 1H), 3.15 (m, 1H), 4.19 (m, 2H), 4.84 (m, 1H), 5.68 (m, 1H), 6.27 (m, 1H), 7.00 (d, 2H), 7.52 (d, 2H), 7.68 (m, 4H).

As a result of observation of the liquid crystal phase of this polymerizable liquid crystal compound (Z4), it was found that the compound converted to an isotropic liquid state at 95° C. and underwent phase transition into a nematic phase at 63° C. during the temperature drop.

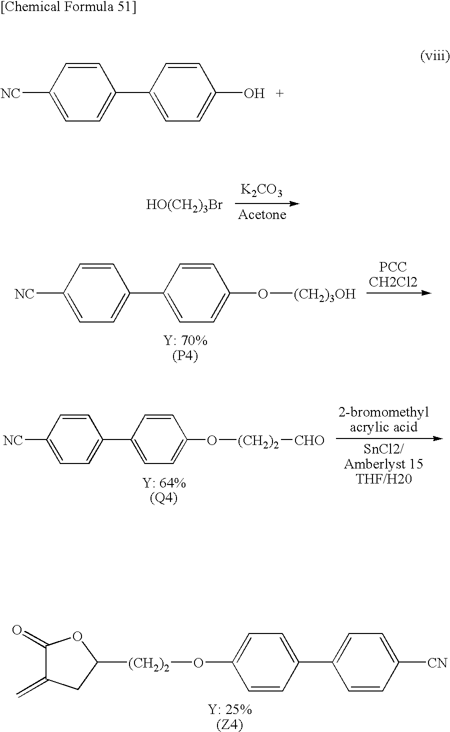

### Example 5

### Synthesis of Polymerizable Liquid Crystal Compound (Z5)

First, 10.0 g (36.4 mmols) of p-(trans-4-heptylcyclo-hexyl)phenol, 8.6 g (36.4 mmols) of 10-bromo-1-decanol, 11.1 g (80 mmols) of potassium carbonate and 150 ml of acetone were added to a 500 ml egg plant-type flask equipped with a condenser tube to provide a mixture, followed by reaction under stirring at 64° C. for 24 hours. After completion of the reaction, the solvent was distilled off under reduced pressure to obtain a light brown solid. Thereafter, this solid and 100 ml of water were mixed, to which 50 ml of diethyl ether was added for extraction. The extraction was repeated three times. The separated organic phase was dried over anhydrous magnesium sulfate, followed by filtration and distilling off the solvent under reduced pressure to obtain a light brown solid.

This solid was dissolved in 5 ml of ethyl acetate and purified with column chromatography (column: silica gel 60 0.063 to 0.200 mm, made by Merck Ltd., eluate: hexane/ethyl acetate=2/1). The solvent was distilled off from the resulting solution to obtain 8.6 g of a light milky-white solid. The results of measurement of the solid with NMR are shown below. From the results, it was confirmed that the light milky-white solid was an intermediate compound (P5) shown in the following synthetic scheme. (Yield: 55%)

¹H-NMR (CDCl₃) δ: 0.89 (t, 3H), 1.10 (m, 2H), 1.26 to 1.65 (m, 29H), 1.83 (m, 6H), 2.40 (m, 1H), 3.63 (t, 2H), 3.93 (t, 2H), 6.81 (d, 2H), 7.11 (d, 2H).

Next, 2.2 g (10.0 mmols) of PCC and 30.0 ml of CH₂Cl₂ were placed in a 200 ml three-necked flask equipped with a condenser tube and mixed with stirring, under which a solution of 4.3 g (10.0 mmols) of the intermediate compound (P5) obtained in the same manner as set out hereinabove dissolved in CH₂Cl₂ (30.0 ml) was dropped, followed by further stirring at 40° C. for 0.5 hour. Subsequently, 90 ml of diethyl ether was added to the solution except for an oily matter attached to the walls of the flask and filtered under reduced pressure, followed by distilling off the solvent under reduced pressure to obtain a dark green, wet solid.

This solid was dissolved in ethyl acetate (3 ml) and purified with silica column chromatography (column: silica gel 60 0.063 to 0.200 mm, made by Merck Ltd., eluate: hexane/ethyl acetate=2/1). The solvent was distilled off from the resulting solution to obtain 3.0 g of a light milky-white solid. The results of measurement of this solid with NMR are shown below. From these results, it was confirmed that this light milky-white solid was an intermediate compound (Q5) shown in the following synthetic scheme (ix). (Yield: 69%)

¹H-NMR (CDCl₃) δ: 0.89 (t, 3H), 1.05 (m, 2H), 1.20 to 1.65 (m, 27H), 1.83 (m, 6H), 2.41 (t, 2H), 3.93 (t, 2H), 6.80 (d, 2H), 7.10 (d, 2H), 9.76 (s, 1H).

Finally, 2.14 g (5.0 mmols) of the intermediate compound (Q5) obtained in the same manner as set out above, 0.8 g (5.0 mmols) of 2-(bromomethyl)acrylic acid, 0.8 g of Amberlyst (registered trade mark) 15 (commercial name of Rohm and Haas Co., Inc.), 8.0 ml of THF, 0.95 g (5.0 mmols) of tin (II) chloride and 2.0 ml of pure water were added to a 50 ml egg plant-type flask equipped with a condenser tube to provide a mixture, followed by reaction under stirring at 70° C. for 24 hours. After completion of the reaction, the reaction solution was filtered under reduced pressure and mixed with 2 ml of pure water, to which 30 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a light brown solid.

This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 1.66 g of a white solid. The results of measurements of the solid with NMR, IR and MS revealed that the white solid consisted of an intended polymerizable liquid crystal compound (Z5) shown in the following synthetic scheme (ix). (Yield: 67%)

The results of the respective measurements are shown below.

¹H-NMR (CDCl₃) δ: 0.87 (t, 3H), 1.05 (m, 2H), 1.20 to 1.45 (m, 29H), 1.87 (m, 6H), 2.40 (m, 1H), 2.60 (m, 1H), 3.03 (m, 1H), 3.93 (t, 2H), 4.52 (m, 1H), 5.61 (m, 1H), 6.22 (m, 1H), 6.81 (d, 2H), 7.12 (d, 2H).

IR (KBr, cm⁻¹): 2921, 1759, 1672.

MS(FAB+): 496(M+)

As a result of observation of the liquid crystal phase of the polymerizable liquid crystal compound (Z5), it was found that the compound converted to an isotropic liquid state at 70° C. and underwent phase transition into a smectic phase at 49° C. during the temperature drop.

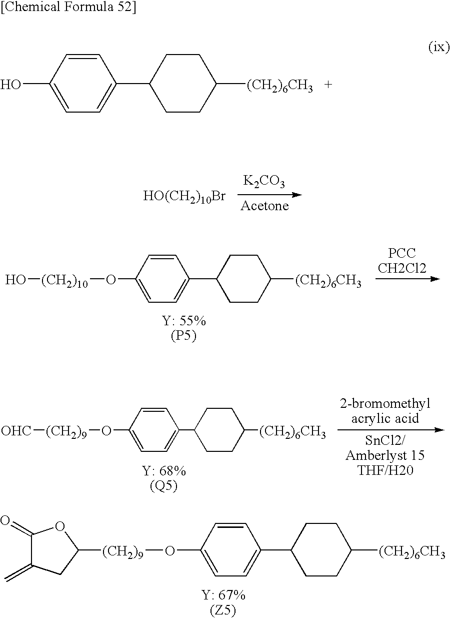

### Example 6

### Synthesis of Polymerizable Liquid Crystal Compound (Z6)

First, 10.0 g (40.6 mmols) of p-(trans-4-pentylcyclo-hexyl)phenol, 7.35 g (40.6 mmols) of 6-bromo-1-hexanol, 11.1 g (80 mmols) of potassium carbonate and 150 ml of acetone were added to a 500 ml egg plant-type flask equipped with a condenser tube to provide a mixture, followed by reaction under stirring at 64° C. for 24 hours. After completion of the reaction, the solvent was distilled off under reduced pressure to obtain a light brown, wet solid. Thereafter, this solid and 100 ml of water were mixed, to which 50 ml of diethyl ether was added for extraction. The extraction was repeated three times. The separated organic phase was dried over anhydrous magnesium sulfate, followed by filtration and distilling off the solvent under reduced pressure to obtain a light brown, wet solid.

This solid was dissolved in 5 ml of ethyl acetate and purified with column chromatography (column: silica gel 60 0.063 to 0.200 mm, made by Merck Ltd., eluate: hexane/ethyl acetate=2/1). The solvent was distilled off from the resulting solution to obtain 11.9 g of a light milky-white solid. The results of measurement of the solid with NMR are shown below. From the results, it was confirmed that the light milky-white solid was an intermediate compound (P6) shown in the following synthetic scheme. (Yield: 86%)

¹H-NMR (CDCl₃) δ: 0.89 (t, 3H), 1.10 (m, 2H), 1.20 to 1.65 (m, 17H), 1.83 (m, 6H), 2.40 (m, 1H), 3.63 (t, 2H), 3.93 (t, 2H), 6.81 (d, 2H), 7.11 (d, 2H).

Next, 4.3 g (20.0 mmols) of PCC and 50.0 ml of CH₂Cl₂ were placed in a 200 ml three-necked flask equipped with a condenser tube and mixed with stirring, under which a solution of 6.9 g (20.0 mmols) of the intermediate compound (P6) obtained in the same manner as set out hereinabove dissolved in CH₂Cl₂ (50.0 ml) was dropped, followed by further stirring at 40° C. for 0.5 hour. Subsequently, 100 ml of diethyl ether was added to the solution except for an oily matter attached to the walls of the flask and filtered under reduced pressure, followed by distilling off the solvent under reduced pressure to obtain a dark green, wet solid.

This solid was dissolved in ethyl acetate (3 ml) and purified with silica column chromatography (column: silica gel 60 0.063 to 0.200 mm, made by Merck Ltd., eluate: hexane/ethyl acetate=2/1). The solvent was distilled off from the resulting solution to obtain 6.0 g of a light milky-white solid. The results of measurement of this solid with NMR are shown below. From these results, it was confirmed that this light milky-white solid was an intermediate compound (Q6) shown in the following synthetic scheme (x). (Yield: 87%)

¹H-NMR (CDCl₃) δ: 0.89 (t, 3H), 1.05 (m, 2H), 1.20 to 1.65 (m, 15H), 1.83 (m, 6H), 2.40 (t, 1H), 2.45 (t, 2H), 3.93 (t, 2H), 6.80 (d, 2H), 7.10 (d, 2H), 9.78 (s, 1H).

Finally, 5.2 g (15.0 mmols) of the intermediate compound (Q6) obtained in the same manner as set out above, 2.5 g (15.0 mmols) of 2-(bromomethyl)acrylic acid, 2.4 g of Amberlyst (registered trade mark) 15 (commercial name of Rohm and Haas Co., Inc.), 24.0 ml of THF, 2.9 g (15.0 mmols) of tin (II) chloride and 6.0 ml of pure water were added to a 50 ml egg plant-type flask equipped with a condenser tube to provide a mixture, followed by reaction under stirring at 70° C. for 24 hours. After completion of the reaction, the reaction solution was filtered under reduced pressure and mixed with 30 ml of pure water, to which 30 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a light brown solid.

This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 3.6 g of a white solid. The results of measurements of the solid with NMR, IR and MS revealed that the white solid consisted of an intended polymerizable liquid crystal compound (Z6) shown in the following synthetic scheme (x). (Yield: 58%)

The results of the respective measurements are shown below.

¹H-NMR (CDCl₃) δ: 0.87 (t, 3H), 1.05 (m, 2H), 1.20 to 1.60 (m, 17H), 1.86 (m, 6H), 2.40 (m, 1H), 2.60 (m, 1H), 3.05 (m, 1H), 3.93 (t, 2H), 4.53 (m, 1H), 5.62 (m, 1H), 6.22 (m, 1H), 6.81 (d, 2H), 7.10 (d, 2H).

IR (KBr, cm⁻¹): 2921, 1758, 1672.

MS(FAB+): 412(M+)

As a result of observation of the liquid crystal phase of the polymerizable liquid crystal compound (Z6), it was found that the compound converted to an isotropic liquid state at 66° C. and underwent phase transition into a smectic phase at 46° C. during the temperature drop.

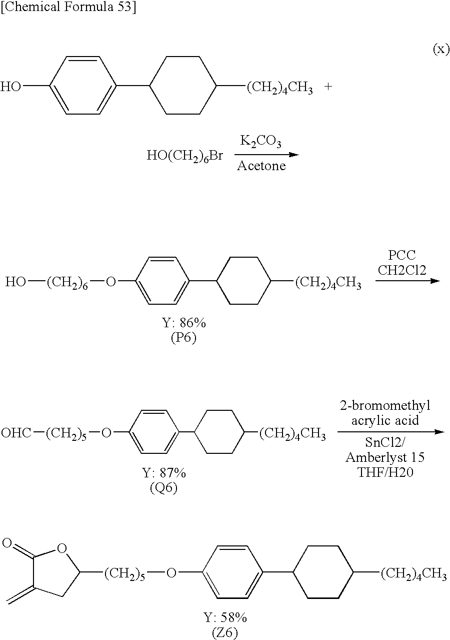

### Example 7

### Synthesis of Polymerizable Liquid Crystal Compound (Z7)

First, 9.0 g (32.7 mmols) of p-(trans-4-heptylcyclo-hexyl)phenol, 5.0 g (32.7 mmols) of 4-bromo-1-butanol, 10.0 g (66.0 mmols) of potassium carbonate and 150 ml of acetone were added to a 500 ml egg plant-type flask equipped with a condenser tube to provide a mixture, followed by reaction under stirring at 64° C. for 48 hours. After completion of the reaction, the solvent was distilled off under reduced pressure to obtain a light brown, wet solid. Thereafter, this solid and 50 ml of water were mixed, to which 30 ml of diethyl ether was added for extraction. The extraction was repeated three times. The separated organic phase was dried over anhydrous magnesium sulfate, followed by filtration and distilling off the solvent under reduced pressure to obtain a light brown, wet solid.

This solid was dissolved in 8 ml of ethyl acetate and purified with column chromatography (column: silica gel 60

Finally, 1.2 g (3.5 mmols) of the intermediate compound (Q7) obtained in the same manner as set out above, 0.58 g (3.5 mmols) of 2-(bromomethyl)acrylic acid, 0.56 g of Amberlyst (registered trade mark) 15 (commercial name of Rohm and Haas Co., Inc.), 5.6 ml of THF, 0.67 g (3.5 mmols) of tin (II) chloride and 1.4 ml of pure water were added to a 50 ml egg plant-type flask equipped with a condenser tube to provide a mixture, followed by reaction under stirring at 70° C. for 7 hours. After completion of the reaction, the reaction solution was filtered under reduced pressure and mixed with 20 ml of pure water, to which 30 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a light brown solid.

This solid was dissolved in 2 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 0.42 g of a light milky-white solid. The results of measurements of the solid with NMR, IR and MS revealed that the light milky-white solid consisted of an intended polymerizable liquid crystal compound (Z7) shown in the following synthetic scheme (xi). (Yield: 29%)

The results of the respective measurements are shown below.

¹H-NMR (CDCl₃) δ: 0.87 (t, 3H), 1.05 (m, 2H), 1.27 (m, 17H), 1.87 (m, 6H), 2.40 (m, 1H), 2.64 (m, 1H), 3.07 (m, 1H), 3.97 (t, 2H), 4.59 (m, 1H), 5.63 (m, 1H), 6.23 (m, 1H), 6.81 (d, 2H), 7.13 (d, 2H).

IR (KBr, cm⁻¹): 2922, 1756, 1664.

MS(FAB+): 412(M+)

As a result of observation of the liquid crystal phase of the polymerizable liquid crystal compound (Z7), it was found that the compound underwent phase transition into a smectic A phase at 66° C. during the temperature rise, converted to an isotropic liquid state at 750° C., and underwent phase transition into a smectic A phase at 72° C. and a smectic X phase at 62° C. both during the temperature drop.

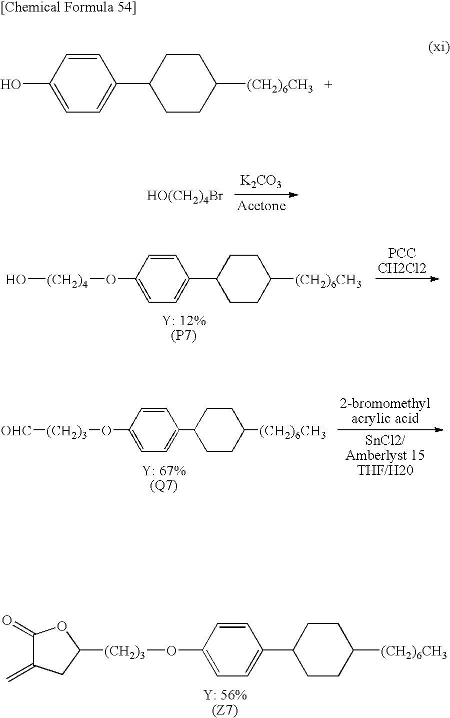

### Example 8

### Synthesis of Polymerizable Liquid Crystal Compound (Z8)

First, 20.0 g (72.9 mmols) of p-(trans-4-heptylcyclo-hexyl)phenol, 10.1 g (72.9 mmols) of 3-bromo-1-propanol, 23.2 g (160.0 mmols) of potassium carbonate and 250 ml of acetone were added to a 500 ml egg plant-type flask equipped with a condenser tube to provide a mixture, followed by reaction under stirring at 64° C. for 48 hours. After completion of the reaction, the solvent was distilled off under reduced pressure to obtain a light brown, wet solid. Thereafter, this solid and 100 ml of water were mixed, to which 60 ml of diethyl ether was added for extraction. The extraction was repeated three times. The separated organic phase was dried over anhydrous magnesium sulfate, followed by filtration and distilling off the solvent under reduced pressure to obtain a light brown, wet solid.

This solid was dissolved in 12 ml of ethyl acetate and purified with column chromatography (column: silica gel 60 0.063 to 0.200 mm, made by Merck Ltd., eluate: hexane/ethyl acetate=2/1). The solvent was distilled off from the resulting solution to obtain 18.3 g of a light milky-white solid. The results of measurement of the solid with NMR are shown below. From the results, it was confirmed that the light milky-white solid was an intermediate compound (P8) shown in the following synthetic scheme. (Yield: 76%)

¹H-NMR (CDCl₃) δ: 0.89 (t, 3H), 1.10 (m, 2H), 1.27 (m, 15H), 1.83 (m, 4H), 2.03 (m, 2H), 2.40 (m, 1H), 3.87 (t, 2H), 4.11 (t, 2H), 6.81 (d, 2H), 7.11 (d, 2H).

Next, 4.3 g (20.0 mmols) of PCC and 50.0 ml of CH₂Cl₂ were placed in a 200 ml three-necked flask equipped with a condenser tube and mixed with stirring, under which a solution of 6.7 g (20.0 mmols) of the intermediate compound (P8) obtained in the same manner as set out hereinabove dissolved in CH₂Cl₂ (50.0 ml) was dropped, followed by further stirring at room temperature for 2 hours. Subsequently, 90 ml of diethyl ether was added to the solution except for an oily matter attached to the walls of the flask and filtered under reduced pressure, followed by distilling off the solvent under reduced pressure to obtain a dark green, wet solid.

This solid was dissolved in ethyl acetate (5 ml) and purified with silica column chromatography (column: silica gel 60 0.063 to 0.200 mm, made by Merck Ltd., eluate: hexane/ethyl acetate=2/1). The solvent was distilled off from the resulting solution to obtain 3.5 g of a light milky-white solid. The results of measurement of this solid with NMR are shown below. From these results, it was confirmed that this light milky-white solid was an intermediate compound (Q8) shown in the following synthetic scheme (xii). (Yield: 53%)

¹H-NMR (CDCl₃) δ: 0.89 (t, 3H), 1.05 (m, 2H), 1.27 (m, 15H), 1.83 (m, 4H), 2.40 (m, 1H), 2.87 (t, 2H), 4.31 (t, 2H), 6.81 (d, 2H), 7.11 (d, 2H), 9.86 (s, 1H).

Finally, 3.3 g (10.0 mmols) of the intermediate compound (Q8) obtained in the same manner as set out above, 1.65 g (10.0 mmols) of 2-(bromomethyl)acrylic acid, 1.6 g of Amberlyst (registered trade mark) 15 (commercial name of Rohm and Haas Co., Inc.), 16.0 ml of THF, 1.90 g (10.0 mmols) of tin (II) chloride and 4.0 ml of pure water were added to a 50 ml egg plant-type flask equipped with a condenser tube to provide a mixture, followed by reaction under stirring at 70° C. for 24 hours. After completion of the reaction, the reaction solution was filtered under reduced pressure and mixed with 30 ml of pure water, to which 50 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a light brown solid.

This solid was dissolved in 3 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 1.92 g of a light milky-white solid. The results of measurements of the solid with NMR, IR and MS revealed that the light milky-white solid consisted of an intended polymerizable liquid crystal compound (Z8) shown in the following synthetic scheme (xii). (Yield: 48%)

The results of the respective measurements are shown below.

¹H-NMR (CDCl₃) δ: 0.87 (t, 3H), 1.05 (m, 2H), 1.28 (m, 15), 1.83 (m, 4H), 2.14 (m, 2H), 2.40 (m, 1H), 2.73 (m, 1H), 3.12 (m, 1H), 4.10 (t, 2H), 4.80 (m, 1H), 5.64 (m, 1H), 6.25 (m, 1H), 6.81 (d, 2H), 7.13 (d, 2H).

IR(KBr, cm⁻¹): 2921, 1762, 1667.

MS(FAB+): 398(M+)

As a result of observation of the liquid crystal phase of the polymerizable liquid crystal compound (Z8), it was found that the compound underwent phase transition into a smectic A phase at 65° C. during the temperature rise, converted to an isotropic liquid state at 77° C., and underwent phase transition into a smectic A phase at 74° C. and a smectic X phase at 44° C. during the temperature drop.

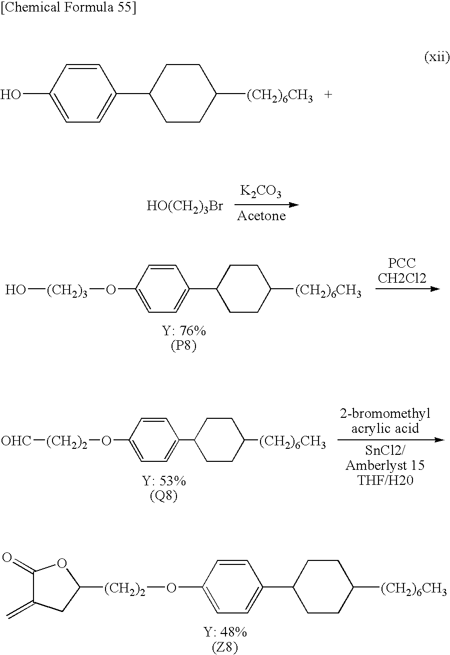

### Example 9

### Synthesis of Polymer (Y1)

First, 0.36 g (1.0 mmol) of a polymerizable liquid crystal compound (Z1) obtained in the same manner as in Example 1, 1.0 ml of dioxane and 2.0 mg of 2,2-azobisiso-butyronitrile (hereinafter referred to as AIBN) were charged into a flask equipped with a condenser tube, followed by reaction under stirring at 70° C. for 24 hours after purging the flask with nitrogen. The resulting reaction solution was charged into 100 ml of pure water, whereupon a white powder precipitated. After filtration, the white powder was vacuum-dried at 40° C. to obtain 0.3 g of polymer (Y1) of the polymerizable liquid crystal compound (Z1) shown in the following polymerization scheme (a1). (Yield: 83%).

The thus obtained polymer had a number average molecular weight of 19300 and a weight average molecular weight of 42400. The polymer was soluble in DMF. The 5 wt % loss temperature of the polymer was at 343° C., with its Tg being at 85° C.

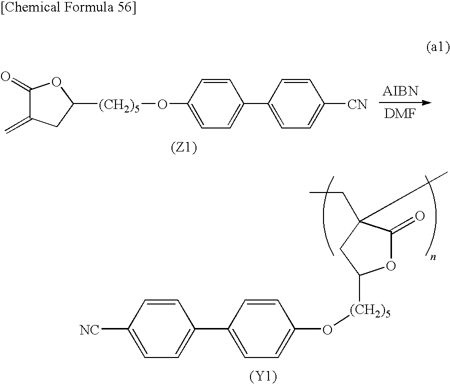

### Example 10

### Synthesis of Polymer (Y2)

First, 1.0 g (2.4 mmols) of a polymerizable liquid crystal compound (Z6) obtained in the same manner as in Example 6, 2.4 ml of dioxane and 4.0 mg of AIBN were charged into a flask equipped with a condenser tube, followed by reaction under stirring at 70° C. for 24 hours after purging the flask with nitrogen. The resulting reaction solution was charged into 400 ml of pure water, whereupon a white powder precipitated. After filtration, the white powder was vacuum-dried at 70° C. to obtain 0.9 g of polymer (Y2) of the polymerizable liquid crystal compound (Z6) shown in the following polymerization scheme (a2). (Yield: 90%).

The thus obtained polymer had a number average molecular weight of 48700 and a weight average molecular weight of 181000. The polymer was soluble in CH₂Cl₂, CH₃Cl, THF and toluene. The 5 wt % loss temperature of the polymer was at 379° C., with its Tg being at 118° C.

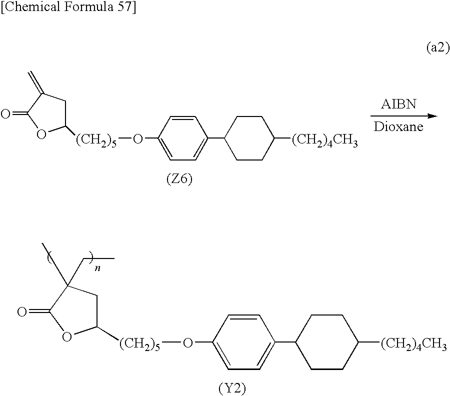

### Example 11

### Synthesis of Polymer (Y3)

First, 0.41 g (1.0 mmol) of a polymerizable liquid crystal compound (Z7) obtained in the same manner as in Example 7, 2.0 ml of dioxane and 2.0 mg of AIBN were charged into a flask equipped with a condenser tube, followed by reaction under stirring at 70° C. for 24 hours after purging the flask with nitrogen. The resulting reaction solution was charged into 100 ml of pure water, whereupon a white powder precipitated. After filtration, the white powder was vacuum-dried at 70° C. to obtain 0.37 g of polymer (Y3) of the polymerizable liquid crystal compound (Z7) shown in the following polymerization scheme (a3). (Yield: 90%)

The thus obtained polymer had a number average molecular weight of 41000 and a weight average molecular weight of 149000. The polymer was soluble in CH₂Cl₂, CH₃Cl, THF and toluene. The 5 wt % loss temperature of the polymer was at 373° C., with its Tg being at 163° C.

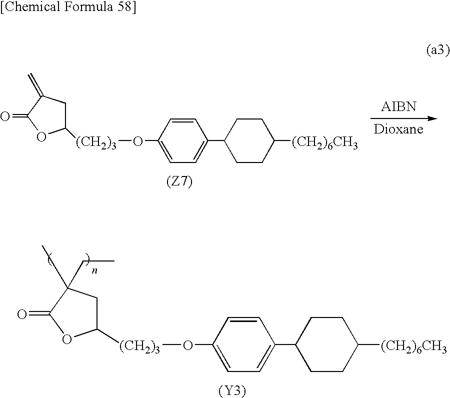

### Example 12

### Synthesis of Polymer (Y4)

First, 0.5 g (1.25 mmols) of a polymerizable liquid crystal compound (Z8) obtained in the same manner as in Example 8, 1.3 ml of dioxane and 2.0 mg of AIBN were charged into a flask equipped with a condenser tube, followed by reaction under stirring at 70° C. for 24 hours after purging the flask with nitrogen. The resulting reaction solution was charged into 100 ml of pure water, whereupon a white powder precipitated. After filtration, the white powder was vacuum-dried at 60° C. to obtain 0.45 g of polymer (Y4) of the polymerizable liquid crystal compound (Z8) shown in the following polymerization scheme (a4). (Yield: 90%)

The thus obtained polymer had a number average molecular weight of 68000 and a weight average molecular weight of 225000. The polymer was soluble in CH₂Cl₂, CH₃Cl, THF and toluene. The 5 wt % loss temperature of the polymer was at 365° C., with its Tg being at 174° C.

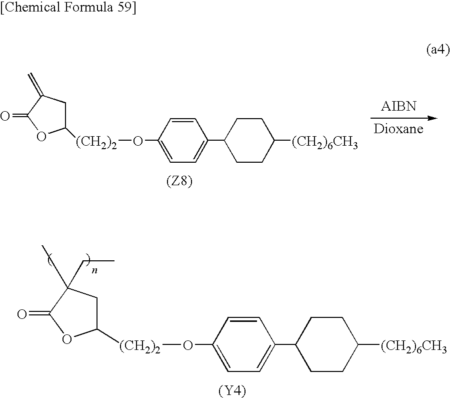

### Example 13

### Polymerizable Liquid Crystal Composition and Polymerized Product (Film) Thereof

First, 50 mg of a polymerizable compound (Z2) obtained in the same manner as in Example 2, 50 mg of a polymerizable compound (Z7) obtained in the same manner as in Example 7 and 50 mg of a polymerizable compound (Z8) obtained in the same manner as in Example 8 were dissolved in 0.5 ml of CH₂Cl₂, and the solvent was distilled off to obtain a colorless solid. 0.150 g of the solid and 1.5 mg of Irgacure (registered trade mark) 369 (commercial name) provided as a photopolymerization initiator and made by Ciba Speciality Chemical Holding, Inc., were mixed to obtain a polymerizable liquid crystal composition.

Next, a substrate attached with a liquid crystal orientation film applied thereon with spacers was heated on a hot plate at 100° C., and the above polymerizable liquid crystal composition was placed on the spacer-applied surface of the substrate. The fluidity of the polymerizable liquid crystal composition was confirmed, after which another liquid crystal orientation film-attached substrate was superposed in contact with the polymerizable liquid crystal composition to provide a cell. The liquid crystal orientation films were ones wherein a liquid crystal orientation agent (SE-1500, made by Nissan Chemical Industries, Ltd.) was spin-coated onto an ITO surface of an ITO-attached glass substrate and baked at 230° C. to provide a 100 nm thick thin film, followed by rubbing. One of the liquid crystal orientation film-attached substrate was applied with silica spacers of 6 μm in size (Shinsikyu SW-D, made by Catalysts and Chemical Co., Ltd.).

The thus obtained cell was irradiated with light whose intensity was at 1600 mJ/cm² by use of a high pressure mercury lamp to polymerize the polymerizable liquid crystal composition. When the cell was observed by means of a polarization microscope with respect to a conoscope image thereof, it was confirmed that the film (polymerized product) in the cell underwent homeotropic alignment as oriented vertically to the glass surface. The retardation value was at 0.28. During the operation, the orientation of the film was maintained.

### Example 14

### Polymerizable Liquid Crystal Composition and Polymerized Product (Film) Thereof

First, 50 mg of a polymerizable compound (Z7) obtained in the same manner as in Example 7 and 50 mg of a polymerizable compound (Z8) obtained in the same manner as in Example 8 were dissolved in 0.5 ml of CH₂Cl₂, and the solvent was distilled off to obtain a colorless solid. Then, 0.100 g of the solid and 1.0 mg of Irgacure (registered trade mark) 369 (commercial name) provided as a photopolymerization initiator and made by Ciba Speciality Chemical Holding, Inc., were mixed to obtain a polymerizable liquid crystal composition.

The polymerizable composition was placed on a cell, which was made by setting two liquid crystal orientation film-attached substrates in such a way that each liquid crystal orientation film turned inside and the rubbing direction was at an angle of 180°, and the cell was heated in a hot plate at 100° C. It was confirmed that the polymerizable composition on the cell turned to a liquid state and was charged into the cell by utilizing a capillary phenomenon. The liquid crystal orientation film-attached substrate used herein was one wherein an ITO-attached glass substrate was spin-coated with a liquid crystal orientation agent (SE-1500, made by Nissan Chemical Industries, Ltd.) at the ITO side thereof and baked at 230° C. to provide a 100 nm thick thin film, followed by rubbing. Another liquid crystal orientation film-attached substrate was attached with a 25 μm thick film spacer along three sides thereof.

The thus obtained cell was irradiated with light whose intensity was at 1600 mJ/cm² by use of a high pressure mercury lamp to polymerize the polymerizable liquid crystal composition. When the cell was observed by means of a polarization microscope with respect to a conoscope image thereof, it was confirmed that the film (polymerized product) in the cell underwent homeotropic alignment as oriented vertically to the glass surface. The transmittance was at 88.4%.

The cell was heated on a hot plate at 100° C. for 10 minutes. The transmittance was at 88.4%. Subsequently, the cell was heated at 120° C. for 10 minutes. The transmittance was at 88.4%. Further, the cell was heated at 150° C. for 10 minutes. The transmittance was 88.3%. During the heatings, the orientation of the film was maintained.

### Example 15

### Polymerizable Liquid Crystal Composition and Polymerized Product (Film) Thereof

A cell was made in the same manner as in Example 14 and evaluated except that the liquid crystal orientation film-attached substrates in Example 14 were replaced by ITO-attached glass substrates and the polymerizable liquid crystal composition was sandwiched between the ITO's.

When the resulting cell was observed by means of a polarization microscope with respect to a conoscope image thereof, it was confirmed that the film (polymerized product) in the cell underwent homeotropic alignment as oriented vertically to the glass surface. The transmittance was at 88.3% and the retardation value was at 0.09.

The cell was heated on a hot plate at 100° C. for 10 minutes. The transmittance was at 88.3%. Subsequently, the cell was heated at 120° C. for 10 minutes. The transmittance was at 88.4%. Further, the cell was heated at 150° C. for 10 minutes. The transmittance was 88.4%. During the heatings, the orientation of the film was maintained.

### Example 16

### Polymerizable Liquid Crystal Composition and Polymerized Product (Film) Thereof

First, 50 mg of a polymerizable compound (Z2) obtained in the same manner as in Example 2, 50 mg of a polymerizable compound (Z7) obtained in the same manner as in Example 7 and 50 mg of a polymerizable compound (Z8) obtained in the same manner as in Example 8 were dissolved in 0.5 ml of CH₂Cl₂, and the solvent was distilled off to obtain a colorless solid. Then, 0.150 g of the solid and 1.5 mg of Irgacure (registered trade mark) 369 (commercial name) provided as a photopolymerization initiator and made by Ciba Speciality Chemical Holding, Inc., were mixed to obtain a polymerizable liquid crystal composition.

This polymerizable composition was placed on a vacant cell made by setting two ITO-attached substrates together so that the ITO faces were turned inside and the cell was heated on a hot plate of 100° C. It was confirmed that the polymerizable composition on the cell turned into a liquid state and was charged into the cell by use of a capillary phenomenon. One of the ITO-attached substrates was attached with a 25 μm thick film spacer along the three sides thereof.

The thus obtained cell was irradiated with light whose intensity was at 1600 mJ/cm² by use of a high pressure mercury lamp at 23° C., thereby polymerizing the polymerizable liquid crystal composition. The resulting cell was observed by means of a polarization microscope with respect to the conoscope image, it was confirmed that the film in the cell underwent homeotropic alignment as oriented vertically to the glass surface. The transmittance was at 88.1% and the retardation value was at 0.33.

The cell was heated on a hot plate at 100° C. for 10 minutes. The transmittance was at 88.3%. Subsequently, the cell was heated at 120° C. for 10 minutes. The transmittance was at 88.4%. Further, the cell was heated at 150° C. for 10 minutes. The transmittance was 88.6%. During the heatings, the orientation of the film was maintained.

### Example 16

### Synthesis of Polymerizable Liquid Crystal Compound (Z9)

First, 25.0 g (91.1 mmols) of p-(trans-4-heptylcyclo-hexyl)phenol, 19.05 g (91.1 mmols) of 5-bromopentyl acetate, 24.9 g (180.0 mmols) of potassium carbonate and 250 ml of acetone were added to a 500 ml egg plant-type flask equipped with a condenser tube to provide a mixture, followed by reaction under stirring at 64° C. for 48 hours. After completion of the reaction, the mixture was filtered and the solvent was distilled off under reduced pressure to obtain a light brown, wet solid.

The solid was dissolved in 100 ml of acetone and while stirring on a water bath at 60° C., 200 ml of ethanol and 50 ml of a 10% NaOH aqueous solution were added. After stirring at 80° C., the acetone was removed from the reaction solution by distilling off to an extent of ¾ thereof, followed by filtration of a white solid.

This solid was purified by recrystallization with ethanol so that 30.6 g of the resulting white solid was obtained. The results of measurement of the solid with NMR are shown below. From the results, it was confirmed that a light milky-white solid was an intermediate compound (P9) shown in the following synthetic scheme (xiv). (Yield: 93%)

¹H-NMR (CDCl₃) δ: 0.89 (t, 3H), 1.10 (m, 2H), 1.26 to 1.65 (m, 19H), 1.83 (m, 6H), 2.40 (m, 1H), 3.68 (t, 2H), 3.93 (t, 2H), 6.81 (d, 2H), 7.11 (d, 2H).

Next, 16.2 g (75.0 mmols) of PCC and 80.0 ml of CH₂Cl₂ were placed in a 500 ml three-necked flask equipped with a condenser tube and mixed with stirring, under which a solution of 27.04 g (75.0 mmols) of the intermediate compound (P2) obtained in the same manner as set out hereinabove dissolved in CH₂Cl₂ (150.0 ml) was dropped, followed by further stirring at a temperature of 40° C. for 0.5 hour. Subsequently, 90 ml of diethyl ether was added to the solution except for an oily matter attached to the walls of the flask and filtered under reduced pressure, followed by distilling off the solvent under reduced pressure to obtain a dark green, wet solid. This solid was dissolved in 3 ml of ethyl acetate and purified with silica column chromatography (column: silica gel 60 0.063 to 0.200 mm, made by Merck Ltd., eluate: hexane/ethyl acetate=2/1). The solvent was distilled off from the resulting solution to obtain 19.3 g of a light milky-white solid. The results of measurement of this solid with NMR are shown below. From these results, it was confirmed that this light milky-white solid was an intermediate compound (Q9) shown in the following synthetic scheme (xiv). (Yield: 75%)

¹H-NMR (CDCl₃) δ: 0.89 (t, 3H), 1.05 (m, 2H), 1.20 to 1.65 (m, 17H), 1.83 (m, 6H), 2.40 (m, 1H), 2.45 (t, 2H), 3.93 (t, 2H), 6.80 (d, 2H), 7.10 (d, 2H), 9.78 (s, 1H).

Finally, 19.3 g (56.0 mmols) of the intermediate compound (Q9) obtained in the same manner as set out hereinbefore, 9.24 g (56.0 mmols) of 2-(bromomethyl)acrylic acid, 9.0 g of Amberlyst (registered trade mark) 15 (commercial name of Rohm and Haas Co., Inc.), 97.0 ml of THF, 10.6 g (56.0 mmols) of tin (II) chloride and 22.0 ml of pure water were added to a 300 ml egg plant-type flask equipped with a condenser tube to provide a mixture, followed by reaction under stirring at 70° C. for 24 hours. After completion of the reaction, the reaction solution was filtered under reduced pressure and mixed with 100 ml of pure water, to which 150 ml of diethyl ether was added for extraction. The extraction was repeated three times. Anhydrous magnesium sulfate was added so as to dry the organic phase obtained after the extraction, followed by filtration under reduced pressure and distilling off the solvent from the solution to obtain a light brown solid. This solid was dissolved in 15 ml of ethyl acetate and purified with silica gel column chromatography (column: silica gel 60 0.063 to 0.200 mm, made by Merck Ltd., eluate: hexane/ethyl acetate=2/1). The solvent was distilled off from the solution to obtain 14.7 g of a white solid. The results of measurements of the solid with NMR and IR revealed that the white solid consisted of an intended polymerizable liquid crystal compound (Z9) shown in the following synthetic scheme (xiv). (Yield: 62%)

The results of the respective measurements are shown below.

¹H-NMR (CDCl₃) δ: 0.87 (t, 3H), 1.05 (m, 2H), 1.20 to 1.80 (m, 19H), 1.84 (m, 6H), 2.40 (m, 1H), 2.60 (m, 1H), 3.06 (m, 1H), 3.93 (t, 2H), 4.54 (m, 1H), 5.61 (m, 1H), 6.23 (m, 1H), 6.81 (d, 2H), 7.12 (d, 2H).

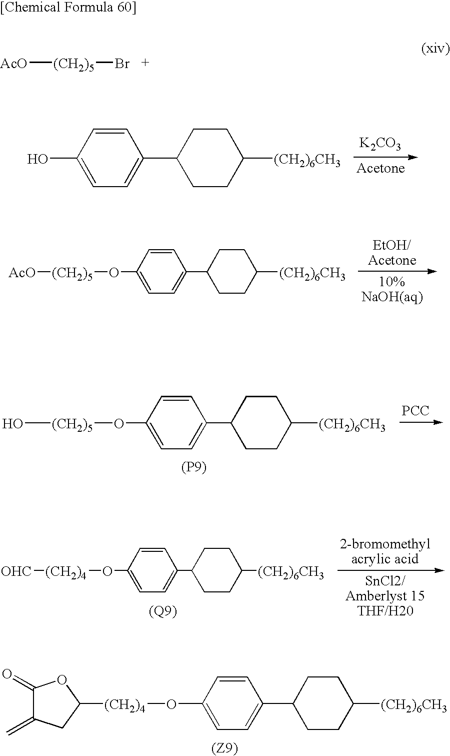

As a result of observation of the liquid crystal phase of the polymerizable liquid crystal compound (Z9), it was found that the compound underwent phase transition into a smectic X phase at 33° C. and a smectic A phase at 51° C. during the temperature rise, converted to an isotropic liquid state at 69° C., and underwent phase transition into a smectic A phase at 65° C. and a smectic X phase (not determined smectic phase) at 49° C. during the temperature drop.

[Reference 1 ]

In a 200 ml three-necked flask, 12.0 g of a compound (P4) obtained in the same manner as in Example 4, 7.7 ml of triethylamine and 0.2 mg of butylhydroxytoluene (BHT) were mixed and dissolved in 40 ml of THF. A solution of 4.6 ml of acrylic acid chloride (acryloyl chloride) dissolved in 40 ml of THF was dropped in the solution under stirring in 15 minutes, during which the three-necked flask was cooled on a water-bath (water temperature: 20° C.). After the dropping, stirring was continued over 30 minutes as it is, after which the flask was removed from the water bath and purged with nitrogen, followed by reaction under further stirring at room temperature for 20 hours. The reaction solution was filtered and the resulting filtrate was concentrated under reduced pressure to ¾ in volume, to which 100 ml of methylene chloride was added. This solution was washed with 100 ml of a saturated sodium carbonate solution, 100 ml of 0.5N hydrochloric acid and 100 ml of a saturated saline solution in this order, followed by drying over magnesium sulfate and distilling off the solvent to obtain a product. This product was recrystallized with ethanol to obtain a polymerizable compound (K1) (6.0 g).

The results of measurement of the polymerizable compound with NMR are shown below. From the results, it was confirmed that the polymerizable compound was a compound (K1) shown in the following synthetic scheme (xv).

¹H-NMR (CDCl₃) δ: 2.20 (m, 2H), 4.10 (t, 2H), 4.40 (t, 2H), 5.81 (d, 1H), 6.15 (m, 1H), 6.41 (d, 1H), 6.99 (d, 2H), 7.55 (d, 2H), 7.66 (m, 4H).

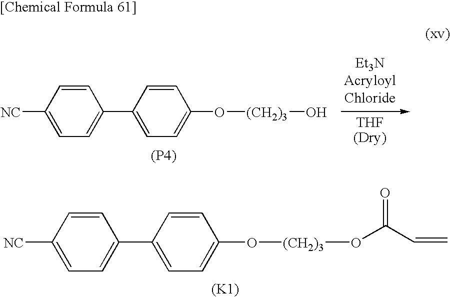

### Example 17

### Polymerizable Liquid Crystal Composition and Polymerized Product (Film) Thereof

First, 100 mg of a polymerizable compound (Z1) obtained in the same manner as in Example 1, 1.0 mg of Irgacure 369 (commercial name) provided as a photopolymerization initiator and made by Ciba-Geigy K. K., and 0.3 mg of FC4430 (made by 3M Co.) of a surfactant were dissolved in 0.4 ml of cyclohexanone to obtain a polymerizable liquid crystal composition. This polymerizable liquid crystal composition was spin-coated onto a liquid crystal orientation film surface of a liquid crystal orientation film-attached substrate (1000 r.p.m., 20 seconds) and prebaked on a hot plate at a temperature of 80° C. for 60 seconds, followed by allowing to cool down to room temperature. At this stage, the polymerizable composition on the substrate was left in liquid crystal state. The liquid crystal orientation film-attached substrate used herein was one wherein a liquid crystal orientation agent (SE-1410, made by Nissan Chemical Industries, Ltd.) was spin-coated onto an ITO surface of an ITO-attached substrate and baked at a temperature of 230° C. to form a 100 nm thick thin film, followed by rubbing.

Next, the coated film formed on the liquid crystal orientation film-attached substrate was irradiated with light whose intensity was at 4000 mJ/cm² in an atmosphere of nitrogen by use of a high pressure mercury lamp to polymerize the polymerizable liquid crystal composition. The resulting film had a thickness of 0.8 μm and when observed through a polarization microscope, it was confirmed that the film oriented horizontally relative to the substrate surface. The transmittance was at 88.6% and the retardation value was at 152 nm.

This film was heated on a hot plate at a temperature of 95° C. for 5 minutes, whereupon the retardation value was at 140 nm. Moreover, upon further heating on a hot plate at a temperature of 95° C. for 5 minutes, the retardation value was at 137 nm and the transmittance was at 88.5%, and the orientation of the film was maintained.

### Example 18

### Polymerizable Liquid Crystal Composition and Polymerized Product (Film) Thereof

First, 30 mg of a polymerizable compound (Z1) obtained in the same manner as in Example 1, 70 mg of compound (K1) obtained in the same manner as in Reference 1, 1.0 mg of Irgacure 369 (commercial name) provided as a photopolymerization initiator and made by Ciba-Geigy K. K., and 0.5 mg of FC4430 (made by 3M Co.) of a surfactant were dissolved in 0.4 ml of cyclohexanone to obtain a polymerizable liquid crystal composition. This polymerizable liquid crystal composition was spin-coated onto a liquid crystal orientation film surface of a liquid crystal orientation film-attached substrate (1000 r.p.m., 20 seconds) and prebaked on a hot plate at a temperature of 80° C. for 60 seconds, followed by allowing to cool down to room temperature. At this stage, the polymerizable composition on the substrate was left in liquid crystal state. The liquid crystal orientation film-attached substrate used herein was one wherein a liquid crystal orientation agent (SE-1410, made by Nissan Chemical Industries, Ltd.) was spin-coated onto the ITO surface of an ITO-attached substrate and baked at a temperature of 230° C. to form a 100 nm thick thin film, followed by rubbing.

Next, the coated film formed on the liquid crystal orientation film-attached substrate was irradiated with light whose intensity was at 4000 mJ/cm² in an atmosphere of nitrogen by use of a high pressure mercury lamp to polymerize the polymerizable liquid crystal composition. The resulting film had a thickness of 0.8 μm and when observed through a polarization microscope, it was confirmed that the film oriented horizontally relative to the substrate surface. The transmittance was at 88.1% and the retardation value was at 126 nm.

This film was heated on a hot plate at a temperature of 95° C. for 5 minutes, whereupon the retardation value was at 140 nm. Moreover, upon further heating on a hot plate at a temperature of 95° C. for 5 minutes, the retardation value was at 30 nm and the transmittance was at 87.9%, and the orientation of the film was maintained.

### Example 19

### Polymerizable Liquid Crystal Composition and Polymerized Product (Film) Thereof

First, 1.00 g of a polymerizable compound (Z7) obtained in the same manner as in Example 7, 1.00 g of a polymerizable compound (Z9) obtained in the same manner as in Example 17, 0.10 g of KAYARAD DPHA (commercial name), made by Nippon Kayaku Co., Ltd., and serving as a specified compound, 20.0 mg of Irgacure 369 (commercial name) provided as a photopolymerization initiator and made by Ciba-Geigy K. K., and 10.0 mg of FC4430 (made by 3M Co.) of a surfactant were dissolved in a mixed solvent of 1.1 mg of butylhydroxytoluene and 8.00 ml of cyclohexanone to obtain a polymerizable liquid crystal composition. This polymerizable liquid crystal composition was spin-coated (1000 rpm, 20 seconds) onto an ITO surface of an ITO-attached substrate and prebaked on a hot plate at a temperature of 60° C. for 60 seconds, followed by allowing to cool down to room temperature. At this stage, the polymerizable composition on the substrate was left in liquid crystal state.

Next, the coated film formed on the ITO-attached substrate was irradiated with light whose intensity was at 4000 mJ/cm² in an atmosphere of nitrogen by use of a high pressure mercury lamp to polymerize the polymerizable liquid crystal composition. The resulting film had a thickness of 1 μm and when observed through a polarization microscope, it was confirmed that the film oriented vertically relative to the substrate surface. The transmittance was at 90.0% and the retardation value was at 0.33 nm.

This film was heated on a hot plate at a temperature of 200° C. for 60 minutes, whereupon the retardation value was at 0.33 nm and the transmittance was at 90.0%, and the orientation of the film was maintained.

### Comparative Example 1

First, 1.17 ml (8.5 mmols) of triethylamine, 2.5 g (7.2 mmols) of an intermediate (P6) obtained in the same manner as in Example 6 and 15.0 ml of dried tetrahydrofuran were changed into a 100 ml three-necked flask equipped with a condenser tube and mixed under stirring in an atmosphere of nitrogen. Subsequently, a solution of 0.82 ml (8.5 mmols) of methacrylic acid chloride dissolved in dried tetrahydrofuran (10.0 ml) was dropped, followed by further stirring at room temperature for 3 hours. After completion of reaction, the reaction solution was filtered under reduced pressure and the solvent was distilled off under the reduced pressure. The residue was dissolved in 50 ml of diethyl ether and washed with 50 ml of distilled water three times, and the resulting reaction product was extracted in an organic phase. Anhydrous magnesium sulfate was added to the organic phase for drying, followed by filtration and distilling off the solvent under reduced pressure to obtain 2.3 g of a colorless oily matter.

The results of measurement of this oily matter with NMR are shown below. From the results, it was confirmed that the oily matter was a compound (R1) shown in the following synthetic scheme (xiii). (Yield: 77%)

¹H-NMR (CDCl₃) δ: 0.86 (t, 3H) 1.05 (m, 2H), 1.15 to 1.95 (m, 26H), 2.39 (m, 1H), 3.93 (m, 2H), 4.13 (m, 2H), 5.54 (s, 1H), 6.09 (s, 1H), 6.81 (d, 2H), 7.09 (d, 2H).

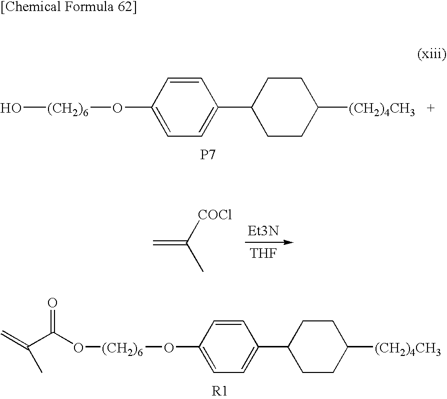

Next, 1.0 g (2.41 mmols) of the compound (R1) obtained above, 2.4 ml of dioxane and 4.0 mg of AIBN were charged into a flask equipped with a condenser tube and the flask was purged with nitrogen, followed by reaction under stirring at 70° C. for 24 hours. The resulting reaction solution was charged into 50 ml of methanol and the resulting precipitated white powder was filtered and vacuum-dried at 40° C. to obtain 0.34 g of a polymer (Y5). (Yield: 34%)

The thus obtained polymer had a number average molecular weight of 10000 and a weight average molecular weight of 24000. The 5 wt % loss temperature of this polymer was 320° C., with Tg being at 62° C.

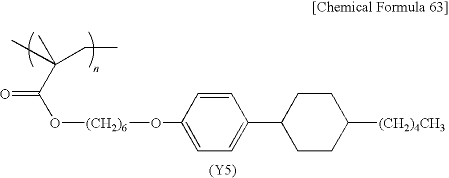

### Comparative Example 2

First, 3.0 mg of a compound (P1) obtained in the same manner as in Example 1, 1.5 ml of triethylamine, 0.1 mg of butylhydroxytoluene (BHT) and 10 ml of THF were mixed and dissolved in a 50 ml three-necked flask. A solution of 0.9 ml of acrylic acid chloride (acryloyl chloride) dissolved in 10 ml of THF was dropped under stirring of the solution in 15 minutes. During the dropping, the three-necked flask was cooled on a water bath (water temperature: 20° C.). After dropping, stirring was continued over 30 minutes, followed by removing the flask from the water bath, purging with nitrogen and reaction under stirring at room temperature for 20 hours. The resulting reaction solution was filtered and the filtrate was concentrated under reduced pressure to an extent of ¾ in volume, to which 50 ml of methylene chloride was added. The resulting solution was washed with 50 ml of a saturated sodium carbonate solution, 50 ml of 0.5N hydrochloric acid and 50 ml of a saturated saline solution in this order, followed by drying over magnesium sulfate and distilling off the solvent to obtain a product. The product was recrystallized from methanol to obtain a polymerizable compound (R2) (1.7 g).

The results of measurement of the polymerizable compound with NMR are shown below. From the results, it was confirmed that the polymerizable compound was a compound (R2) shown in the following synthetic scheme (xvii).

¹H-NMR (CDCl₃) δ: 1.50 (m, 4H), 1.73 (m, 2H), 1.85 (m, 2H), 4.05 (t, 2H), 4.20 (t, 2H), 5.82 (d, 1H), 6.15 (m, 1H), 6.41 (d, 1H), 6.99 (d, 2H), 7.55 (d, 2H), 7.66 (m, 4H).

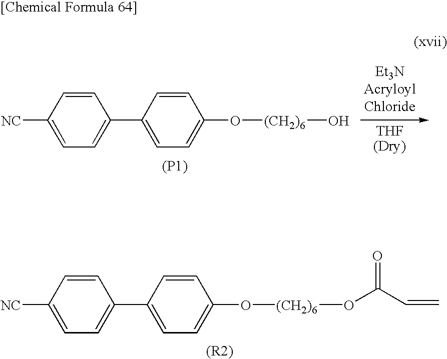

### Comparative Example 3

First, 50 mg of the polymerizable compound (R2) obtained in the same manner as in Comparative Example 2, 50 mg of the compound (K1) obtained in the same manner as in Reference 1, 1.0 mg of Irgacure 369 (commercial name) provided as a photopolymerization initiator and made by Ciba-Geigy K. K. and 0.5 mg of FC4430 of a surfactant were dissolved in 0.4 ml of cyclohexanone to obtain a composition.

The composition was spin-coated onto a liquid crystal orientation film surface of a liquid crystal orientation film-attached substrate (1000 r.p.m., 20 seconds) and prebaked on a hot plate at a temperature of 80° C. for 60 seconds, followed by allowing to cool down to room temperature. The composition on the substrate was left in liquid crystal state. The liquid crystal orientation film-attached substrate used herein was one wherein a liquid crystal orientation agent (SE-1410, made by Nissan Chemical Industries, Ltd.) was spin-coated onto an ITO surface of a ITO-attached glass substrate and beaked at a temperature of 230° C. to form a 100 nm thick thin film, followed by rubbing.

Next, the film formed on the liquid crystal orientation film-attached substrate was irradiated with light whose intensity was at 4000 mJ/cm² in an atmosphere of nitrogen by use of a high pressure mercury lamp to polymerize the composition. The resulting film had a thickness of 0.7 μm and when observed with a polarization microscope, the film oriented horizontally relative to the substrate surface. The transmittance was at 87.1% and the retardation value was at 124 nm.

The film was heated on a hot plate at a temperature of 95° C. for 5 minutes, whereupon the retardation value was at 3 nm and the transmittance was at 89.2%, and the disturbance in orientation was observed in the film.

### INDUSTRIAL APPLICABILITY

The polymerizable liquid crystal composition including a polymerizable compound according to the invention is useful as a material for an optical compensation film such as a polarizer plate or a phase difference plate for display devices and an optically anisotropic film such as a multi-domain film or the like.

1. A polymerizable liquid crystal compound, characterized by being represented by the following formula [1] 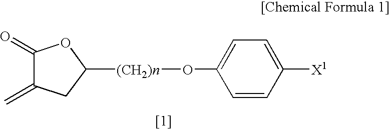
in the formula [1], n is an integer of 2 to 9 and X¹ is of the formula [2] or [3] 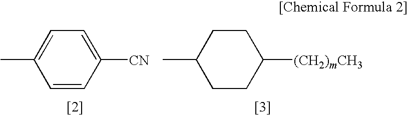
in the formula [3], m is an integer of 4 to 8.

2. The polymerizable liquid crystal composition comprising at least one of polymerizable liquid crystal compounds defined in claim 1.

3. The polymerizable liquid crystal composition comprising at least one of polymerizable liquid crystal compounds defined in claim 1, and a polymerizable liquid crystal compound of a type other than the first-mentioned polymerizable liquid crystal compounds.

4. The polymerizable liquid crystal composition according to claim 3, wherein the polymerizable liquid crystal compound of other type is represented by the following formula [5] 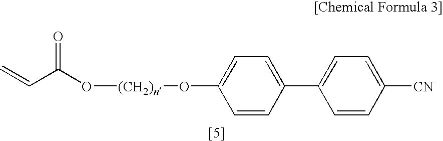
in the formula [5], n′ is an integer of 1 to 6.

5. The polymer obtained by polymerizing the polymerizable liquid crystal compound defined in claim 1.

6. The polymer obtained by polymerizing a polymerizable liquid crystal composition defined in any one of claims 2 to 4.

7. A film obtained by using the polymerizable liquid crystal compound defined in claim 1.

8. A film obtained by using a polymerizable liquid crystal composition defined in any one of claims 2 to 4.
